(19) United States
(12) Patent Application Publication
(10) Pub. No.: US 2010/0087370 A1
(43) Pub. Date: Apr. 8, 2010

(54) MODULATION OF NITRIC OXIDE SIGNALING TO NORMALIZE TUMOR VASCULATURE

(75) Inventors: Rakesh K. Jain, Wellesley, MA (US); Dai Fukumura, Newton, MA (US); Satoshi Kashiwagi, Boston, MA (US)

Correspondence Address: EDWARDS ANGELL PALMER & DODGE LLP, P.O. BOX 55874, BOSTON, MA, 02205, US

(73) Assignee: THE GENERAL HOSPITAL CORPORATION, Boston, MA (US)

(21) Appl. No.: 12/525,186

(22) PCT Filed: Feb. 14, 2008
(86) PCT No.: PCT/US08/02004

§ 371 Date: Nov. 30, 2009

Related U.S. Application Data
(60) Provisional application No. 60/901,144, filed on Feb. 14, 2007.

(51) Int. Cl. A61K 38/18; A61K 31/7088; A61P 35/00

(52) U.S. Cl. 514/12; 514/44 R

(57) ABSTRACT

The instant invention provides methods for treating a solid tumor in a subject comprising modulating nitric oxide production in the tumor to normalize tumor vasculature and administering an anti-tumor therapy to the subject. The invention further provides methods of treating a solid tumor in a subject comprising selectively increasing cyclic guanosine monophosphate (cGMP) or cGMP dependent protein kinase G production in the tumor vasculature to an amount effective to normalize tumor vasculature and administering an anti-tumor therapy to the subject.

### RELATED APPLICATIONS/PATENTS & INCORPORATIONS BY REFERENCE

This application claims priority to U.S. provisional patent application Ser. No. 60/901,144, filed Feb. 14, 2007, the entire disclosure of which is incorporated herein by this reference.

### STATEMENT OF GOVERNMENT SUPPORT

The work leading to the present invention was funded in part by grant number R01CA096915 and P01CA80124 from the United States National Institutes of Health. Accordingly, the United States Government has certain rights to this invention.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

Tumor vessels are structurally and functionally abnormal, with defective endothelium, basement membrane and pericyte coverage (Carmeliet and Jain, 2000 Nature 407, 249-257; Dvorak, 2002 J. Clin. Oncol. 20, 4368-4380). These abnormalities impair the delivery of oxygen and therapeutics (Jain, 2003 Nat. Med. 7, 987-989). In theory, reducing or abolishing vascular abnormalities by anti-angiogenic therapy should “normalize” the tumor vasculature and alleviate hypoxia (Jain, 2001 Nat. Med. 9, 685-693). On the other hand, extensive destruction of tumor vessels by anti-angiogenic therapy can also hinder the delivery of oxygen and drugs, as reported in cases where the anti-angiogenic agent TNP-470 was combined with radiation (Murata et al., 1997 Int. J Radiat. Oncol. Biol. Phys. 37, 1107-1113) and chemotherapy (Ma et al., 2001 Cancer Res 61, 5491-5498). It is therefore important to determine how to combine these therapies to produce optimal therapeutic effects.

Clinical and experimental data suggest that nitric oxide plays a role in promoting solid tumor growth and progression (Fukumura, et al. 2006 Nature Reviews Cancer 6:521-534). For example, nitric oxide generation by inducible nitric oxide synthase (iNOS) has been implicated in the development of prostate cancer (Klotz et al. Cancer; National Library of Medicine, MDX Health Digest 1998 82(10):1897-903), as well as in colonic adenocarcinomas and mammary adenocarcinomas (Lala, P. K. and Orucevic, A., Cancer and Metastasis Reviews 1998 17:91-106). In addition, nitric oxide has been suggested to play an important role in the metabolism and behavior of lung cancers, and in particular adenocarcinomas (Fujimoto et al. Jpn. J. Cancer Res 1997 88: 1190-1198). In fact, it has been suggested that tumor cells producing or exposed to what these researchers refer to as low levels of nitric oxide, or tumor cells capable of resisting nitric oxide-mediated injury undergo a clonal selection because of their survival advantage (Lala, P. K. and Orucevic, A. Cancer and Metastasis Review 1998 17:91-106). It has been suggested that these tumor cells utilize certain nitric oxide-mediated mechanisms for promotion of growth, invasion, and metastasis and been proposed that nitric oxide-blocking drugs may be useful in treating certain human cancers. There is also evidence indicating that tumor-derived nitric oxide promotes tumor angiogenesis, as well as invasiveness of certain tumors in animals, including humans (Lala, P. K. Cancer and Metastasis Reviews 1998 17:1-6).

### BRIEF SUMMARY OF THE INVENTION

In one aspect, the invention provides a method of treating a solid tumor in a subject, the method comprising the steps of: modulating nitric oxide production in the tumor to normalize tumor vasculature; and administering an anti-tumor therapy to the subject, thereby treating the solid tumor in the subject.

In one embodiment of the invention, the solid tumor is a glioblastoma.

In another embodiment of the invention, the nitric oxide production is selectively increased in the tumor vasculature.

In yet another embodiment of the invention, the nitric oxide production is selectively increased in the tumor vasculature by administering an agent that increases the expression of endothelial nitric oxide synthase to the tumor vasculature of the subject.

In yet another embodiment of the invention, the nitric oxide production is selectively increased in the tumor vasculature by administering an agent that increases the activity of endothelial nitric oxide synthase to the tumor vasculature of the subject. The agent that increases the activity of endothelial nitric oxide synthase may not be expressed in the tumor. The agent can be a peptide selected from the group consisting of, but not limited to, a vascular endothelial growth factor, angiopoietin-1, platelet derived growth factor-beta, transforming growth factor-beta, estrogen, BH4: (6R)-5,6,7,8-tetrahydro-L-biopterin, RANKL, an inhibitor of caveolin-1 and bradykinin. The agent that increases the activity of endothelial nitric oxide synthase can likewise be selected from the group consisting of, but not limited to, a statin, L-arginin, calcium ionophore, sphingosine-1-phosphate, nitrite and acethylcholine. The statin can be selected from the group consisting of, but not limited to, Atorvastatin, Cerivastatin, Fluvastatin, Lovastatin, Mevastatin, Pravastatin, Rosuvastatin and Simvastatin.

In specific embodiments of the invention, the agent is administered by intravenous delivery or in a cationic delivery vehicle that is greater than about 100 nm.

In yet another embodiment of the invention, the nitric oxide production is selectively increased in the tumor vasculature by providing low dose radiation in a range of between about 2 Gy to about 6 Gy to the subject.

In still further embodiments of the invention, the nitric oxide production is selectively increased in the tumor vasculature by administering nitric oxide synthase, preferably an endothelial nitric oxide synthase, to the tumor vasculature of the subject. The nitric oxide synthase can be administered, for example, in a cationic delivery vehicle that is greater than about 100 nm. The endothelial nitric oxide synthase can also be administered by intravenous delivery.

In yet another embodiment of the invention, the nitric oxide production is selectively increased in the tumor vasculature by administering an expression vector comprising a nucleic acid sequence encoding a nitric oxide synthase, preferably an endothelial nitric oxide synthase, to the tumor vasculature of the subject. The nucleic acid sequence encoding a nitric oxide synthase can be expressed, for example, by an endothelial specific promoter. In yet another embodiment, the expression vector is administered by intravenous delivery.

In yet another embodiment of the invention, the nitric oxide production is selectively increased in the tumor vasculature by administering a nitric oxide donor to the tumor vasculature of the subject. The nitric oxide donor can be selected from the group consisting of, but not limited to, a DETANONOate, GEA, SNAP, GSNO, ISDN, NOC, NOR, Spermine NONOate, NO-donating nonsteroidal anti-inflammatory drugs (NO-NSAIDs), nitrite and S-nitorosohemoglobin. In still further embodiments, the nitric oxide donor can be administered by intravenous delivery or in a cationic delivery vehicle that is greater than about 100 nm.

In yet another embodiment of the invention, non-vascular cells of the tumor produce nitric oxide and said nitric oxide production is selectively decreased in said cells. The nitric oxide production can be selectively decreased by, for example, administering an inhibitor of inducible nitric oxide synthase. The inhibitor of inducible nitric oxide synthase can be selected from the group consisting of, but not limited to, a aminoguanidine, 1400 W, L-NIL, GW273629, GW274150, ITU, tryptanthrin, steroid, non-steroidal anti-inflammatory, inhibitor of NRkB, inhibitor of IL-1, inhibitor of TNF, and inhibitor of IFN-gamma. The inhibitor of inducible nitric oxide synthase may, in a further embodiment, comprise an expression vector comprising a nucleic acid sequence encoding an inducible nitric oxide synthase interfering RNA or antisense RNA under the control of a tumor specific promoter. The interfering RNA can be, for example, an RNAi or shRNA.

In a further embodiment, the nitric oxide production is selectively decreased by administering an inhibitor of neuronal nitric oxide synthase. The inhibitor of neuronal nitric oxide synthase can be selected from the group consisting of, but not limited to, a L-NPA, 7-nitroindazole, ARL 17477, Vinyl-L-NIO and TRIM. In still a further embodiment, the inhibitor of neuronal nitric oxide synthase may comprise an expression vector comprising a nucleic acid sequence encoding a neuronal nitric oxide synthase interfering RNA or antisense RNA under the control of a tumor specific promoter. The interfering RNA can be for example, an RNAi or shRNA.

In another aspect, the invention further comprises monitoring tumor vasculature to detect normalized tumor vasculature prior to administering the anti-tumor therapy to the subject. The normalized tumor vasculature can, for example, be detected by identifying a normal basement membrane in the tumor vasculature or by identifying perivascular cell recruitment to the tumor vasculature or by measuring a parameter of the tumor vasculature selected from the group consisting of vessel density, vessel diameter, vessel brunching and vessel tortuosity or by measuring permeability of the tumor vasculature or by measuring blood flow of the tumor vasculature or by measuring interstitial fluid pressure of the tumor tissue or by measuring oxygenation of the tumor tissue or by detecting delivery of an agent within the tumor tissue.

In various embodiments of the invention, the tumor treated by methods of the invention is a solid tumor selected from the group consisting of, but not limited to, a adrenocortical carcinoma, epithelial carcinoma, desmoid tumor, desmoplastic small round cell tumor, endocrine tumor, Ewing sarcoma family tumor, germ cell tumor, hepatoblastoma, hepatocellular carcinoma, lymphoma, melanoma, neuroblastoma, non-rhabdomyosarcoma soft tissue sarcoma, osteosarcoma, peripheral primitive neuroectodermal tumor, retinoblastoma, rhabdomyosarcoma, and Wilms tumor.

In one embodiment of the invention, the growth of the tumor is reduced. In another embodiment of the invention, the tumor is eradicated.

In yet another embodiment of the invention, the anti-tumor therapy is radiation.

In yet another embodiment of the invention, the anti-tumor therapy is a cytotoxic agent. The cytotoxic agent can be, for example, a chemotherapeutic agent selected from the group consisting of, but not limited to, a 5-FU, vinblastine, actinomycin D, etoposide, cisplatin, methotrexate, doxorubicin, ganciclovir, 4-[(2-chloroethyl)(2-mesyloxyethel)amino] benzoyl-L-glutamic acid, cyclophosphamide and busulphan.

In yet another embodiment of the invention, the anti-tumor therapy is an immune activator selected from the group consisting of an interferon, interleukin, tumor necrosis factor, granulocyte-macrophage colony-stimulating factor, and Fins-like tyrosine kinase ligand 3.

In yet another embodiment of the invention, the anti-tumor therapy is an expression vector comprising a nucleic acid sequence encoding a herpes simplex virus thymidine kinase, cytosine deaminase, carboxypeptidase, p 53, multiple drug resistance gene-1, anti-sense bcl-2, anti-sense c-myc, anti-sense K-ras, and anti-sense c-erbB2.

In yet another aspect, the invention provides a method of reducing the growth of a solid tumor in a subject, the method comprising the steps of: selectively increasing nitric oxide production in the tumor vasculature to an amount effective to normalize tumor vasculature; decreasing nitric oxide production in the non-vascular tumor cells; and administering an anti-tumor therapy to the subject, thereby reducing the growth of the solid tumor in the subject.

In yet another aspect, the invention provides a method of treating a solid tumor in a subject, the method comprising the steps of: selectively increasing cyclic guanosine monophosphate (cGMP) production in the tumor vasculature to an amount effective to normalize tumor vasculature; and administering an anti-tumor therapy to the subject, thereby treating the solid tumor in the subject. In another embodiment of the invention, the cGMP production is selectively increased in the tumor vasculature by administering an agent that increases the activity of soluble guanylyl cyclase to the tumor vasculature of the subject. The agent can be selected from the group consisting of, but not limited to, a nitric oxide, YC-1, natridiuretic peptide, BAY 41-2272, BAY 41-8543 and BAY 58-2667. In further embodiments of the invention, the agent is administered by intravenous delivery or in a cationic delivery vehicle that is greater than about 100 nm.

In yet another embodiment of the invention, the cGMP production in the tumor is selectively increased in the tumor vasculature by administering a phosphodiesterase inhibitor to the tumor vasculature of the subject. The phosphodiesterase inhibitor can be, for example, selected from the group consisting of, but not limited to, a sildenafil, vardenafil, sulindac sulfone, NCX-911, T-0156, JNJ-10258859, FR226807, Tadalafil, T-1032, SCH51866, Win65579, DMPPO, and 1-arylnaphthalene.

In yet another aspect, the invention provides a method of treating a solid tumor in a subject, the method comprising the steps of: selectively increasing cGMP dependent protein kinase G activity or expression in the tumor vasculature to an amount effective to normalize tumor vasculature; and administering an anti-tumor therapy to the subject, thereby treating the solid tumor in the subject. In further embodiments of the invention, the cGMP dependent protein kinase G is cGMP dependent protein kinase G1 or cGMP dependent protein kinase G2.

In another embodiment of the invention, the cGMP dependent protein kinase G activity is selectively increased in the tumor vasculature by administering an agent that increases cGMP dependent protein kinase G activity to the tumor vasculature of the subject. The agent can be, for example, cGMP. In still further embodiments of the invention, the agent is administered by intravenous delivery or in a cationic delivery vehicle that is greater than about 100 nm.

In yet another embodiment of the invention, non-vascular cells of the tumor have cGMP dependent protein kinase G activity or expression and said activity or expression is selectively decreased in said cells.

In yet another embodiment of the invention, the cGMP dependent protein kinase G activity or expression is selectively decreased by administering an inhibitor of said activity or expression.

In yet another aspect, the invention provides a method of increasing the bioavailability of an anti-tumor therapy within a solid tumor, the method comprising the steps of: modulating nitric oxide production in the tumor to normalize tumor vasculature; and administering an anti-tumor therapy to the tumor, thereby increasing the bioavailability of the anti-tumor therapy within the solid tumor.

In yet another aspect, the invention provides a method for normalizing tumor vasculature in a solid tumor of a subject, comprising selectively increasing nitric oxide production in the tumor vasculature to an amount effective to increase the amount of perivascular cells within abnormal blood vessels of the tumor vasculature, thereby normalizing tumor vasculature in the solid tumor of the subject.

Other aspects of the invention are described in the following disclosure and are within the ambit of the invention.

### BRIEF DESCRIPTION OF THE FIGURES

The following Detailed Description, given by way of example, but not intended to limit the invention to specific embodiments described, may be understood in conjunction with the accompanying figures, incorporated herein by reference.

FIGS. 1A-C show analysis of parental U87MG and nNOS shRNA-transfected U87 tumors by immunoassays. (A) Immunohistochemical staining of cultured parental U87MG tumors grown in cranial windows. (B) Immunoblots from Western blot analysis of cultured parental U87MG and nNOS shRNA-transfected U87 cells. (C) Immunoblots from Western blot analysis of in vivo tumors grown in cranial windows.

FIGS. 2A-B show in graph form, the growth kinetics of parental and nNOS silencing U87 glioma. (A) Bar graph depicting tumor size (fold increase) at 10 days compared to 1 day after implantation for U87 tumors grown in cranial window. (B) Plot depicting tumor volume, as determined by a caliper, for U87 tumors (n=5 each) grown subcutaneously.

FIGS. 3A-B show microfluorography depicting NO distribution in U87 gliomas grown in the cranial window in Rag-1−/− NO production was visualized by means of DAF-2T fluorescence 0, 20, 40 and 60 min after DAF-2 (0.5 mg/body) injection. Left column, micro-angiography with tetramethylrhodamine-dextran (2000 kDa). Middle column, representative DAF-2T microfluorography captured 60 min after the loading of DAF-2 in tumors. Right column, pseudocolor representation of DAF-2T microfluorography. Color bar on the right shows calibration of the fluorescence intensity with known concentrations of DAF-2T (DAF-2Tapp). Bars indicate 100 μm. (A) NO distribution in parental U87MG (top row) or nNOS shRNA58-transfected-U87 (bottom row) tumors. (B) NO distribution in U87 tumors of the animals were treated with a control compound D-NMMA (top row) or L-NMMA, an inhibitor of all NOS isoforms (bottom row).

FIGS. 4A-C show the effect of nNOS silencing on blood vessel morphology in U87 gliomas. (A) MPLSM microangiograms depicting U87MG, U87-shRNA58-, and U87-shNA150-transfected tumors. Images were taken by multiphoton laser scanning microscopy following FITC-dextran (2,000 kDa) i.v. injection. Images are 630 μm across and 2-D projection of 200 μm thickness. Quantification of 3-D vessel morphology: (B) in bar graph form, vascular density in U87, U87-shNA58, and U87-shRNA150 tumors; and (C) in bar graph form, vessel diameter in U87, U87-shNA58, and U87-shRNA150 tumors. n=10, 6, and 4 for U87MG, U87-shRNA58, and U87-shRNA150, respectively. *p<0.05, as compared to U87MG tumors.

FIGS. 5A-C show the effect of nNOS silencing in U87 gliomas on perivascular cell coverage and vascular permeability. (A) Immunohistochemical analysis of perivascular cell coverage. Histological specimens of parental and nNOS silencing U87, with αSMA-positive perivascular cells (red) and biotinylated lectin-stained vascular endothelial cells (blue) in perfused blood vessels identified. The bar indicates 20 μm. (B) In bar graph form, percentage of vessel perimeter covered with αSMA-positive perivascular was quantified in 15-20 sections of each tumor type. * P<0.05 as compared to U87MG tumors. (C) Microvascular permeability in U87MG (N=11), U87-shRNA58 (N=8) and U87-shRNA150 (N=5) tumors was measured by intravital microscopy using tetramethylrhodamine-BSA. * P<0.05 as compared to U87MG tumors.

FIGS. 6A-D show the effect of an nNOS selective inhibitor on vessel morphology and function in U87 tumors. An nNOS selective inhibitor, L-NPA (20 mg/kg, daily i.p. injection) was used to block nNOS activity pharmacologically. (A) MPLSM microangiographies of U87MG tumors grown in the cranial window in SCID mice treated with saline and L-NPA. The bar indicates 200 μm. (B-C) Quantification of 3-D vessel density (B) and diameter (C) using MPLSM image stack and an automated image analysis system. N=5 each. (D) Microvascular permeability in U87MG tumors treated with saline (N=6) and L-NPA (N=5) was measured by intravital microscopy using tetramethylrhodamine-BSA. * P<0.05 as compared to saline treated groups.

FIGS. 7A-E show NOS expression in GL261 tumors and the effect of an nNOS selective inhibitor on vessel morphology and function in GL261 tumors. (A) NOS expression in GL261 tumors grown in the cranial window in SCID mice. Five micron paraffin block sections were immunostained using antibodies to eNOS, nNOS, iNOS or non-specific mouse IgG. The bar indicates 100 μm. Different from U87MG tumors, GL261 tumor cells express all three isoforms of NOS, especially strong expression of eNOS. (B) MPLSM microangiographies of GL261 tumors treated with saline and L-NPA. The bar indicates 200 μm. (C-D) Quantification of 3-D vessel density (C) and diameter (D) using MPLSM image stack and an automated image analysis system. N=6 each. (E) Microvascular permeability in GL261 tumors treated with saline (N=6) and L-NPA (N=6) was measured by intravital microscopy using tetramethylrhodamine-BSA. L-NPA treatment did not alter vessel morphology and function in GL261 tumors.

FIGS. 8A-D show the effects of pan-NOS inhibition and eNOS inhibition on U87MG tumor vessels. (A) Representative microangiography images of U87MG tumors grown in the cranial window in SCID mice treated with D-NMMA (N=9) and L-NMMA (N=12). The bar indicates 100 μm. (B) Vessel parameters quantified by off-line analyses of the digitized microangiography images. * P<0.05 as compared with D-NMMA group. (C) Representative microangiography images of U87MG tumors grown in the cranial window in SCID mice treated with control compound AP (N=8) and an eNOS selective inhibitor cavtratin (N=11). The bar indicates 100 μm. (D) Vessel parameters quantified by off-line analyses of the digitized microangiography images. * P<0.05 as compared with AP group.

FIGS. 9A-E show the effect of nNOS silencing in U87 gliomas on tumor tissue oxygenation. Tissues shown in (A) were harvested and stained after injection of pimonidazole (60 mg/kg), followed by biotinylated lectin. Confocal laser scanning microscopy images (top row) of Hypoxyprobe™-1 adducts stained hypoxic cells (red), lectin-bound perfused blood vessels (green) and nuclei (blue) in U87MG, U87-shRNA58, and U87-shRNA58. Images are 630 μm across. Binarized images (bottom row) of above confocal images. Bar indicates 100 μm. (B-C) Quantification of vessel segments (B) and vessel perimeter (C). (D) Quantification of hypoxic area. n=7, 3, 4 respectively. * p<0.05, as compared to U87MG tumors. (E) Western blot analysis of HIF-1α expression in U87MG and nNOS-shRNA-transfected U87 tumors. HIF-1α protein levels in U87-shRNA58 and U87-shRNA150 tumors were 46% and 59% of that in U87MG tumors, respectively.

FIGS. 10A-C show the effect of nNOS silencing in U87 gliomas on fractionated radiation therapy. (A) Tumor growth normalized to day 0 tumor volume. (B) Tumor growth delay evaluated at the levels of 2, 4, and 6 times V₀. * P<0.05 as compared to U87MG. (C) Kaplan-Meier survival plot. U87 control (n=9), sh58 control (n=7), sh150 control (n=9), U87 radiation (n=8), sh58 radiation (n=6), sh150 radiation (n=10).

FIGS. 11A-D show in vitro radiosensitivity and post-radiation tumor vasculature in U87 tumors. (A) Intrinsic tumor cell radiosensitivity was evaluated with the clonogenic assay. Following irradiation, the cells were incubated 9-13 d for colony formation depending on the dose administered. The surviving fractions were corrected for initial and final multiplicities determined 4-6 h after plating and at the time of irradiation. Data are expressed as mean±s.d. (B-D) When U87MG and nNOS-silenced U87 tumors grown in the hindleg in Rag-1^{-/-}-mice reached ˜100 mm³ they received 8 Gy/d for 3 d. One day after the completion of radiation, tumor tissues were harvested following the administration of biotinylated lectin and then stained. (B) Confocal laser scanning microscopy images of lectin-bound perfused blood vessels (green) and DAPI stained nuclei (blue). Bar indicates 100 μm. (C-D) Quantification of the number of vessel segments (C) and vessel perimeter (D). N=3, 4, 4 for U87MG, shRNA58 and shRNA150. * P<0.05 as compared to U87MG tumors.

FIGS. 12A-E show the effect of iNOS inhibition in MCaIV tumors and blood vessel morphology and function in MCaIV tumors. (A) Fluorescence immunohistochemistry image of iNOS expression (green), F4/80 positive macrophages (red) and DAPI stained nuclei (blue) in MCaIV tumors grown in the mammary fat pad. The bar indicates 50 μm. (B-C) MPLSM images of MCaIV tumors grown in αSMA^{P}-GFP mice treated with saline control (B) or an iNOS inhibitor 1400 W (C). Functional blood vessels were contrast-enhanced by i.v. injection of tetramethylrhodamine-dextran (red). αSMA-positive perivascular cells were visualized by GFP fluorescence (green). The bars indicate 100 μm. (D-E) MCaIV tumors grown in αSMAP-GFP mice were treated with saline (control) or iNOS inhibitor (1400 W) for 7 days. (D) Perivascular cell coverage. (N=4, each). (E) Vascular permeability (N=7, each). Data are mean±SEM. *p<0.05 vs. control.

FIGS. 13A-C show the role of the NO-sGC-cGMP pathway in perivascular cell recruitment and migration. (A) In bar graph form, cGMP production in cultured 10T1/2 cells in response to NO donor or PDE5 inhibitor. (B) In bar graph form, the migration of 10T1/2 cells in a transwell assay. 10T1/2 cell migration was assessed using Falcon HTS FluoroBlok inserts with 1 μm pores. GFP-expressing 10T1/2 cells were inoculated in the inserts, and HUVECs were inoculated in the outer well. Percent area of transwell filter covered by migrated 10T1/2 cells was determined. Medium only indicates no HUVECs in the outer well. ODQ, T-1032, Sildenafil, or Sildenafil+L-NMMA were added to the medium. * P<0.05 vs. control (HUVEC). (C) Images for transmigrated GFP-10T1/2 cells at the back side of the Fluoroblock insert after 10 hours with control vs. T-1032 treatment.

FIGS. 14A-B show the effect of PI3K inhibition on transwell migration of 10T1/2 cells toward HUVECs. 10T1/2 cell migration was assessed using Falcon HTS FluoroBlock inserts with 1 μm pores. GFP-expressing 10T1/2 cells were inoculated in the inserts and human umbilical vein endothelial cells (HUVECs) were inoculated in the outer well. (A) Images for transmigrated GFP-10T1/2 cells at the back side of the FluoroBlock insert after 16 hours. Images are 865 μm across. (B) Quantification of 10T1/2 cell transwell migration. Data are expressed as percentage relative to the control (HUVEC) migration of the same experimental batch. Medium represents no HUVECs in the outer well. LS294002 10 μM was added. n=4 each. * p<0.05 as compared to the control (HUVEC).

FIGS. 15A-C show expression of sGCβ1 in various tissues. (A) Expression of sGCβ1 in mouse liver; (B) Expression of sGCβ1 in B16F10 tumors. Bar indicates 100 μm; (C) Expression of sGCβ1 in U87 tumors. Bar indicates 50 μm.

### DETAILED DESCRIPTION OF THE INVENTION

### I. Definitions

Unless otherwise defined, all technical and scientific terms used herein have the same meaning as commonly understood by one of ordinary skill in the art to which this invention belongs. In case of conflict, the present application, including definitions will control.

As used herein “anti-tumor therapy” refers to any therapy to decrease tumor growth or metastasis, including surgery, radiation, and/or chemotherapy.

As used herein, a “cytotoxic agent” refers to any agent capable of destroying cells, preferably dividing cells such as cancer cells.

As used herein “an increase in activity” of a nitric oxide synthase enzyme refers to an increase in the activity of the enzyme in catalyzing the oxidation of L-arginine to L-citrulline and nitric oxide (NO), i.e., providing an increased production of NO, in a subject. Thus, “increased activity” means that a NOS enzyme activity that is greater than the activity in the subject before treatment. “Increased”, then, refers to an amount of NO production at least about 1-fold more than (for example 1, 2, 3, 4, 5, 10, 20, 30, 40, 50, 60, 70, 80, 90, 100, 1000, 10,000-fold or more) the amount of NO production in a subject before treatment. “Increased” as it refers to NOS enzyme activity also means at least about 5% more than (for example 5, 6, 7, 8, 9, 10, 15, 20, 25, 30, 35, 40, 45, 50, 55, 60, 65, 70, 75, 80, 85, 90, 95, 99 or 100%) the amount of activity (i.e., NO production) in a subject before treatment. Nitric oxide synthase enzyme activity (NO production) can be measured by methods known in the art.

As used herein “an increase in expression” of a nitric oxide synthase enzyme refers to an increase in the mRNA or protein expression of the nitric oxide synthase gene in a subject. Thus, “increased expression” refers to an amount of NOS expression at least about 1-fold more than (for example 1, 2, 3, 4, 5, 10, 20, 30, 40, 50, 60, 70, 80, 90, 100, 1000, 10,000-fold or more) than the amount of NOS expression in a subject prior to treatment. “Increased” as it refers to the amount of NOS expression in a subject also means at least about 5% more than (for example 5, 6, 7, 8, 9, 10, 15, 20, 25, 30, 35, 40, 45, 50, 55, 60, 65, 70, 75, 80, 85, 90, 95, 99 or 100%) the amount of NOS expression in the subject before treatment. Expression of NOS enzyme can be measured according to methods known in the art.

As used herein “an interfering RNA” refers to any double stranded or single stranded RNA sequence, capable—either directly or indirectly (i.e., upon conversion)—of inhibiting or down regulating gene expression by mediating RNA interference. Interfering RNA includes but is not limited to small interfering RNA (“siRNA”) and small hairpin RNA (“shRNA”). “RNA interference” refers to the selective degradation of a sequence-compatible messenger RNA transcript.

As used herein “an shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA by a cleavage event mediated by the enzyme Dicer, which is a member of the RNase III family.

As used herein “an RNAi” (RNA interference) refers to a post-transcriptional silencing mechanism initiated by small double-stranded RNA molecules that suppress expression of genes with sequence homology.

As used herein “nitric oxide donor” refers to a variety of NO donors including, but not limited to, organic NO donors, inorganic NO donors and prodrug forms of NO donors, “NO prodrugs”, “NO producing agents”, “NO delivering compounds”, “NO generating agents”, and “NO providers”.

As used herein “nitric oxide mimetic” refers to nitric oxide, or a functional equivalent thereof; any compound which mimics the effects of nitric oxide, generates or releases nitric oxide through biotransformation, generates nitric oxide spontaneously, or spontaneously releases nitric oxide; any compound which in any other manner generates nitric oxide or a nitric oxide-like moiety or activates other stages of the NO pathway; or any compound which enables or facilitates NO utilization by the cell, when administered to an animal. Such compounds can also be referred to as “NO donors”. Examples of such compounds include, but are not limited to: organonitrates such as nitroglycerin (GTN), isosorbide mononitrates (ISMN) which include isosorbide 2-mononitrate (IS2N) and/or isosorbide 5-mononitrate (ISSN), isosorbide dinitrate (ISDN), pentaerythritol tetranitrate (PETN), erythrityl tetranitrate (ETN); ethylene glycol dinitrate, isopropyl nitrate, glyceryl-1-mononitrate, glyceryl-1,2-dinitrate, glyceryl-1,3-dinitrate, butane-1,2,4-triol trinitrate, and S-nitrosoglutathione (SNOG); compounds that serve as physiological precursors of nitric oxide, such as L-arginine, L-citrulline and salts of L-arginine and L-citrulline; and other compounds which generate or release NO under physiologic conditions such as S,S-dinitrosodithiol (SSDD), [N-[2-(nitroxyethyl)]-3-pyridinecarboxamide (nicorandil), sodium nitroprusside (SNP), hydroxyguanidine sulfate, N,O-diacetyl-N-hydroxy-4-chlorobenzenesulfonamide, S-nitroso-N-acetylpenicilamine (SNAP), 3-morpholino-sydnonimine (SIN-1), molsidomine, DEA-NONOate(2-(N,N-diethylamino)-diazenolate-2-oxide), (*)-(E)-ethyl-2-[(E)-hydroxyimino]-5-nitro-3-hexeneamide, (*)-N-[(E)-4-ethyl-3-[(Z)-hydroxyimino]-5-nitro-3-hexen-1-yl]-3-pyridinec-carboxamide, 4-hydroxymethyl-3-furoxancarboxamide and spermine NONOate (N-[4-[1-(3-aminopropyl)-2-hydroxy-2-nitrosohydrazino]butyl-1,3-propanediamine). Organic nitrates GTN, ISMN, ISDN, ETN, and PETN, as well as nicorandil (commonly known as a potassium channel opener) are commercially available in pharmaceutical dosage forms. SIN-1, SNAP, S-thioglutathione, spermine NONOate, and DEA-NONOate are commercially available from Biotium, Inc. Richmond, Calif.

As used herein the term “nitric oxide mimetic” is also intended to mean any compound which acts as a nitric oxide pathway mimetic, that has nitric oxide-like activity, or that mimics the effect of nitric oxide. Such compounds may not necessarily release, generate or provide nitric oxide, but they have a similar effect to nitric oxide on a pathway that is affected by nitric oxide. For example, nitric oxide has both cyclic GMP-dependent and cyclic GMP-independent effects. Nitric oxide is known to activate the soluble form of guanylyl cyclase, thereby increasing intracellular levels of the second messenger cyclic GMP and other interactions with other intracellular second messengers such as cyclic AMP. As such, compounds which directly activate either particulate or soluble guanylyl cyclase such as natriuretic peptides (ANP, BNP, and CNP), 3-(5′-hydroxymethyl-2′furyl)-1-benzyl indazole (YC-cGMP or YC-1) and 8-(4-chlorophenylthio)guanosine 3′,5′-cyclic monophosphate (8-PCPT-cGMP), are also examples of NO-mimetics.

Nitric oxide mimetic activity encompasses those signal transduction processes or pathways which comprise at least one NO mimetic-binding effector molecule, such as for example, guanylyl cyclase and other heme containing proteins. Example of agents which function as NO mimetics by enabling or facilitating NO utilization by the cell are compounds which inhibit phosphodiesterase activity and/or expression, such as phosphodiesterase inhibitors.

As used herein “inhibitor” of a nitric oxide synthase enzyme refers to a compound that decreases, as defined herein, or otherwise interferes with, for example modifies or changes, the activity or expression of iNOS and/or nNOS under normal or disease conditions. That is, an inhibitor or antagonist of iNOS and/or nNOS decreases either (iNOS and/or nNOS) activity or expression as compared to activity or expression in the absence of the inhibitor or antagonist. The inhibitor can have a direct or indirect effect on iNOS and/or nNOS. For example, an inhibitor that decreases iNOS and/or nNOS activity may do so by interacting with an iNOS and/or nNOS ligand.

As used herein “a selective increase” in nitric oxide production within the tumor vasculature refers to an increase in nitric oxide production that occurs in the vessels of the tumor but not within the non-vascular tumor tissue and/or stroma.

As used herein, a “solid tumor” refers to an abnormal mass of tissue that usually does not contain cysts or liquid areas. Solid tumors may be benign (not cancerous), or malignant (cancerous). Generally, a solid tumor connotes cancer of body tissues other than blood, bone marrow, or the lymphatic system.

As used herein “tumor specific promoter” refers to a promoter that permits gene expression specifically in tumor cells, and not in the tumor vasculature. The promoter and coding sequence are operatively linked so as to permit transcription of the sequence encoding the gene.

As used herein, “endothelial specific promoter” refers to a promoter that permits gene expression specifically in endothelial cells, for example, the vascular endothelial (VE) cadherin gene promoter.

As used herein, a “subject” refers to any member of the class mammalia, including humans, domestic and farm animals, and zoo, sports or pet animals, such as mouse, rabbit, pig, sheep, goat, cattle and higher primates.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. Treatment of a solid tumor includes, but is not limited to, inhibiting tumor growth, inhibiting tumor cell proliferation, reducing tumor volume, or inhibiting the spread of tumor cells to other parts of the body (metastasis).

As used herein “tumor vasculature” refers to blood vessels (arteries, capillaries, veins) transporting blood towards and away from a tumor (i.e., the tumor's blood supply). The tumor vasculature consists of both vessels coopted from the preexisting network of the host (subject) vasculature and vessels resulting from the angiogenic response of host vessels to cancer cells (Jain, R. K. 2001 J of Controlled Release 74:7-25).

As used herein “non-vascular tumor cells” refer, collectively, to interstitial and surrounding cells of the tumor, and exclude cells comprising the tumor blood vessels. Non-vascular tumor cells include, without limitation, stromal cells such as fibroblasts, and immune cells.

As used herein “vascular normalization” refers to a physiological state during which existing tumor vessels exhibit improved structure in the vascular endothelium and basement membrane and therefore, have reduced hypoxia.

As used herein, the terms “comprises,” “comprising,” “containing” and “having” and the like are open-ended as defined by U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

### II. Methods of the Invention

### Tumor Vessel Normalization

Unlike normal blood vessels, tumor vessels are structurally and functionally abnormal, with defective endothelium, basement membrane and pericyte coverage (Carmeliet and Jain, 2000 Nature 407, 249-257; Dvorak, 2002 J. Clin. Oncol. 20, 4368-4380). An imbalance of pro- and antiangiogenic factors causes endothelial cell migration and proliferation. The excess endothelial cells and abnormal perivascular cells contribute to the formation of tortuous, dilated, and saccular blood vessels that are poorly organized and hyperpermeable (Jain, R. K. 2001 Nature Med 7(9):987-989). These abnormalities, as well as the compression of blood vessels by cancer cells, can increase resistance to blood flow and impair blood supply. As a result, the delivery and effectiveness of conventional cytotoxic therapies, as well as molecular targeted therapies, are compromised.

If immature and inefficient blood vessels could be pruned by eliminating excess endothelial cells, the resulting vasculature would be more “normal” and, hence, more conducive to the delivery of nutrients and drugs. Because an abnormal vasculature poses a formidable challenge to the delivery of nutrients and drugs to solid tumors, normalization of the abnormal (tumor) vasculature (for example, by restoring the balance of pro- and antiangiogenic cytokines) can facilitate the delivery of therapeutics to tumors.

### Nitric Oxide Signaling

There are three isoforms of nitric oxide synthase (NOS). Endothelial nitric oxide synthase (eNOS, also referred to as type III NOS) is constitutively expressed by vascular endothelial cells. It has a calcium-dependent activity and generates relatively low levels of NO. The NO produced by eNOS mediates a variety of physiological functions in vivo including neovascularization, regulation of blood vessel tone (vessel wall tension), platelet aggregation, vascular permeability, and leukocyte-endothelial interaction (Moncada, S. 1992 Acta Physiol Scand 145:201-227; Fukumura, D., et al. 1998 Cell 94:715-725).

By contrast, inducible nitric oxide synthase (iNOS, type II NOS) is transcriptionally regulated by inflammatory cytokines and other stimuli. It is calcium-independent, and it generates higher levels of NO (than does eNOS), which can induce cytostatic or toxic effects. Finally, neuronal nitric oxide synthase (nNOS, type I NOS) mediates the transmission of neuronal signals.

Recent data have revealed the predominant role of endothelial nitric oxide synthase (eNOS) in both angiogenesis (the development of new blood vessels derived from existing vessels) and vasculogenesis (blood vessel formation de novo from progenitor cells). NO that is predominantly synthesized by eNOS in vascular endothelial cells promotes angiogenesis directly and functions both upstream and downstream of angiogenic stimuli. Moreover, NO mediates recruitment of perivascular cells and, therefore, remodeling and maturation of blood vessels. NO that is synthesized by eNOS promotes tumor progression through the maintenance of blood flow, induction of vascular hyperpermeability, and reduction of leukocyte-endothelial interaction.

It is contemplated herein that the modulation of eNOS constitutes a viable strategy for controlling pathological neovascularization. A selective increase in eNOS (activity or expression), optionally coupled with a decrease in iNOS and/or nNOS, promotes normalization of tumor vasculature.

There are multiple growth factors, which mediate vessel maturation. The importance of cytokine gradients from vascular endothelial cells has also been shown for platelet derived growth factor-B induced perivascular cell recruitment and integration in the vessel wall (Klamer et al., 2004 European Journal of Pharmacology 503:103-107). NO has also been shown to mediate the function of many angiogenic factors such as vascular endothelial growth factor, angiopoietin-1 and sphingosine-1-phosphate (Gratton et al., 2003 Cancer Cell 4:31-39; Tyrrell et al., 2007 IEEE Transactions on Medical Imaging 26:223-237; Fukumura et al., 2001 Proc Natl Acad Sci USA 98:2604-2609. Additionally, NO can induce the expression of endogenous angiogenic factors such as vascular endothelial growth factor and basic fibroblast growth factor (Winkler et al. 2004 Cancer Cell 6:553-563; Hranitzky et al., 1973 Radiology 107:641-644). It is conceivable that there is local crosstalk or coordination between NO and other growth factors during vascular morphogenesis and vessel maturation.

eNOS activity or expression can be selectively increased via administration into the tumor vasculature (e.g., preferably selective administration into the tumor vasculature) of an agent including, without limitation, a NO mimetic, a NO donor (such as DETANONOate (see J. A. Hrabie, et al. (1993) J. Org. Chem. 58, 1472 and L. K. Keefer, et al. (1996) Meth. Enzymol. 268, 281), GEA (see J. Robak, et al. (1995) Pharmacol. Res. 25 S2, 355 and E. Moilanen, et al. (1993) Br. J. Pharmacol. 109, 852), SNAP (see Donors of Nitrogen Oxides, Chapter 7: M. Feelisch & J. S. Stamler; Methods in Nitric Oxide Research, 71, eds. M. Feelisch & J. S. Stamler (John Wiley & Sons, Inc., 1996, B. Roy, et al. (1994) JOC 59, 7019, J. Ramirez, et al. (1996) Bioorg. Med. Chem. Lett. 6, 2575, and Y. Hou, et al. (1999) Meth. Enzymol. 301, 242), GSNO (see Chapter 7: M. Feelisch & J. S. Stamler; Methods in Nitric Oxide Research, 71, eds. M. Feelisch & J. S. Stamler (John Wiley & Sons, Inc., 1996) and S-nitroso-glutathione inhibits platelet activation in vitro and in vivo: M. W. Radomski, et al. (1992) Br. J. Pharmacol. 107, 745), ISDN (also known as isosorbide dinitrate, see T. Taylor, et al. (1982) Arzneimittelforschung 32, 1329 and S. D. Maletic, et al. (1999) Physiol. Res. 48, 417), NOC (see J. A. Hrabie & J. R. Klose (1993) JOC 58, 1472), NOR (see Y. Kita, et al. (1994) Eur. J. Pharmacol. 257, 123), Spermine NONOate (see L. K. Keefer, et al. (1996) Meth. Enzymol. 268, 281), NO-donating nonsteroidal anti-inflammatory drugs (NO-NSAIDs), nitrite, or S-nitorosohemoblobin), a peptide (such as vascular endothelial growth factor, angiopoietin-1, platelet derived growth factor-beta, transforming growth factor-beta, estrogen, BH4: (6R)-5,6,7,8-tetrahydro-L-biopterin, RANKL, an inhibitor of caveolin-1, or bradykinin), an expression vector comprising a nucleic acid sequence encoding eNOS, a statin (such as Atorvastatin, Cerivastatin, Fluvastatin, Lovastatin, Mevastatin, Pravastatin, Rosuvastatin, or Simvastatin), L-arginin, calcium ionophore, sphingosine-1-phosphate, nitrite and acetylcholine.

The presence or absence of vascular normalization can be identified by detecting, for example, the return of tumor vessel diameter to the smaller diameter that is typically present in a normal host tissue. Alternatively, functional parameters of the tumor vasculature can be monitored to detect changes associated with normalization. These parameters include, but are not limited to, vessel permeability and basement membrane thickness. It can, thus, be determined whether, for example, the modulation of nitric oxide production induces vascular normalization by measuring the effects of selectively increasing NO production in the tumor vasculature. Standard dosages known in the art for the agents that increase eNOS activity or expression can be administered, or if needed, can be adjusted to an amount effect to normalize tumor vasculature by routine variation according to the results observed with the detection methods.

Furthermore, the “window of normalization” (i.e., the point at which a suitable portion of the tumor vasculature is normalized) following, for example, the selective increase of NO production can be detected. Upon detection of the window of normalization, an anti-tumor therapy can be administered to the subject to reduce the growth of or eradicate the solid tumor.

INOS and/or nNOS can be decreased via administration of an agent including, without limitation, i) aminoguanidine, 1400 W (also known as N-(3-(Aminomethyl)benzyl) acetamidine, see Garvey, E P, et al., (1997) J Biol. Chem. 272(8):4959-63), L-NIL (also known as L-N6-(1-iminoethyl)lysine, see Moore, W M, et al., (2004) J Med Chem. 37(23):3886-8), GW273629 (also known as (3-[[2-[(1-iminoethyl)amino]ethyl]sulphonyl]-L-alanine), see Alderton W K, et al, (2005) Br J Pharmacol. 145(3):301-12), GW274150 (also known as ([2-[(1-iminoethyl) amino]ethyl]-L-homocysteine), see Alderton W K, et al, (2005) Br J Pharmacol. 145(3):301-12), ITU (also known as isothiourea, see Garvey, E P, et al., (1994) J Biol Chem. October 28; 269(43):26669-76), tryptanthrin, steroid, non-steroidal anti-inflammatory, inhibitor of NRkB, inhibitor of IL-1, inhibitor of TNF, inhibitor of IFN-gamma, and an expression vector comprising a nucleic acid sequence encoding an inducible nitric oxide synthase interfering RNA (such as RNAi or shRNA) or antisense RNA under the control of a tumor specific promoter; and ii) L-NPA (also known as N-omega-propyl-L-arginine, see H. Q. Zhang, et al. (1997) J. Med. Chem. 40, 3869), 7-nitroindazole (see P. A. Bland-Ward & P. K. Moore (1995) Life Sci. 57, PL131), ARL 17477 (see Zheng G Zhang et al. (1996) Journal of Cerebral Blood Flow & Metabolism 16, 599-604), TRIM (also known as 1-(2-trifluoromethylphenyl) imidazole, see R. L. Handy, et al. (1995) Br. J. Pharmacol. 116, 2349), Vinyl-L-NIO (also known as N5-(1-Imino-3-butenyl)-L-ornithine, see B. R. Babu & O. W. Griffith; (1998) J. Biol. Chem. 273, 8882) and an expression vector comprising a nucleic acid sequence encoding a neuronal nitric oxide synthase interfering RNA (such as RNAi or shRNA) or antisense RNA under the control of a tumor specific promoter.

Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule (i.e., iNOS or nNOS) or a portion thereof and the sense strand comprises nucleotide sequence corresponding to the target nucleic acid sequence or a portion thereof.

Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

Modulating Cyclic Guanosine Monophosphate (cGMP) Production and cGMP Dependent Protein Kinase Activity or Expression

Many of the physiological processes that are promoted by NO are mediated by the NO-cGMP signaling pathway. In this pathway, NO, endogenously produced by NO synthases or released from exogenously applied NO donors, activates NO-sensitive (soluble) guanylyl cyclase (GC) and leads to increased synthesis of cyclic guanosine monophosphate (cGMP). Elevated cGMP activates cGMP-dependent protein kinase (PKG), leading to decreased intracellular calcium concentration ([Ca²⁺]ᵢ) and subsequent relaxation. The resulting vasodilation increases blood flow in the affected vascular bed (Lincoln, T. M., et al. 1996 Biochemistry of Smooth Muscle Contraction, New York: Academic Press, p. 257-268).

It is contemplated herein that a selective increase in cGMP production and/or cGMP protein kinase g (PKG) activity or expression in the tumor vasculature of a subject results in some degree of normalization of the tumor vasculature. Administration of anti-tumor therapy in concert with such a selective increase effects treatment of a solid tumor in the subject. cGMP production in tumor vasculature can be selectively increased by, for example, administering an agent (such as nitric oxide, YC-1 (see F. N. Ko, et al. (1994) Blood 84, 4226), natridiuretic peptide, BAY 41-2272 (see J. P. Stasch, et al. (2001) Nature 410, 212, E. M. Becker, et al. (2001) BMC Pharmacol. 1, 13, A. Straub, et al. (2001) Bioorg. Med. Chem. Lett. 11, 781 and M. Koglin, et al. (2002) BBRC 292, 1057), BAY 41-8543 (see Stasch J P, et al. (2002) British Journal of Pharmacology. 135(2):344-55, and Stasch J P, et al., (2002) British Journal of Pharmacology. 135(2):333-43) or BAY 58-2667 (see Stasch J P. et al. (2002) British Journal of Pharmacology. 136(5):773-83) that increases the activity of soluble guanylyl cyclase or administering a phosphodiesterase inhibitor (such as sildenafil, vardenafil, sulindac sulfone, NCX-911, T-0156, JNJ-10258859, FR226807, Tadalafil, T-1032, SCH51866, Win65579, DMPPO, or 1-arylnaphthalene) to the tumor vasculature of the subject. For a discussion of stimulators and activators of soluble guanylate cyclase see, Evgenov O V, et al., (2006) Nature Reviews. Drug Discovery. 5(9):755-68.

CGMP protein kinase G activity or expression in tumor vasculature can be selectively increased by, for example, administration into the tumor vasculature (e.g., preferably selective administration into the tumor vasculature) of an agent (such as cGMP) that increases cGMP dependent protein kinase G activity to the subject's tumor vasculature.

### Tumors

Types of tumors to be treated are preferably solid tumors including, without limitation, sarcomas, carcinomas and other solid tumor cancers, including, but not limited to germ line tumors, tumors of the central nervous system, breast cancer, prostate cancer, cervical cancer, uterine cancer, lung cancer, ovarian cancer, testicular cancer, thyroid cancer, astrocytoma, glioma, glioblastoma, pancreatic cancer, stomach cancer, liver cancer, colon cancer, melanoma, renal cancer, bladder cancer, esophageal cancer, cancer of the larynx, cancer of the parotid, cancer of the biliary tract, rectal cancer, endometrial cancer, squamous cell carcinomas, adenocarcinomas, small cell carcinomas, neuroblastomas, mesotheliomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, lymphomas, melanomas, non-rhabdomyosarcoma soft tissue sarcomas, osteosarcomas, peripheral primitive neuroectodermal tumors, retinoblastomas, rhabdomyosarcomas, Wilms tumors, and the like.

Reduction of tumor growth means a measurable decrease in growth of the tumor of at least about 0.01-fold (for example 0.01, 0.1, 1, 3, 4, 5, 10, 100, 1000-fold or more) or decrease by at least about 0.01% (for example 0.01, 0.1, 1, 3, 4, 5, 10, 20, 30, 40, 50, 60, 70, 80, 90, 95, 99 or 100%) as compared to the growth measured over time prior to treatment as defined herein.

Full eradication of the tumor may also be achieved through methods of the invention. Eradication refers elimination of the tumor. The tumor is considered to be eliminated when it is no longer detectable using detection methods known in the art (e.g., imaging).

### Anti-tumor Therapy

Contemplated herein as anti-tumor therapy administered to the subject being treated for a solid tumor according to a method of the invention (in addition to the agent or therapy effecting modulation of nitric oxide production) are, without limitation, surgery, radiation, chemotherapy, cytotoxic agents, and immune activators.

Cytotoxic agents include chemotherapeutic agents, radiation therapy, and anti-angiogenic agents. The cytotoxic agent can be a chemical agent, such as a chemotherapeutic agent used in cancer treatment (adriamycin or etoposide, for example) or hormones such as tamoxifen or other biologicals such as TNF-a or bFGF. In one embodiment, the anti-angiogenic agent modulates a vascular endothelial growth factor receptor, such as vascular endothelial growth factor receptor-2, by blocking the receptor. For example, the anti-angiogenic agent can be an antibody, such as DC101, Avastin and Herceptin.

The anti-angiogenic agent can also be, but is not limited to, Endostatin, Angiostatin, Galardin (GM6001, Glycomed, Inc., Alameda, Calif.), low molecular weight VEGF receptor kinases (e.g., Novartis PTK787 and AstraZeneca ADZ2171), endothelial response inhibitors (e.g., agents such as interferon alpha, TNP470, and vascular endothelial growth factor inhibitors), agents that prompt the breakdown of the cellular matrix (e.g., Vitaxin (human LM-609 antibody, Ixsys Co., San Diego, Calif.; Metastat, CollaGenex, Newtown, Pa.; and Marimastat BB2516, British Biotech), agents that act directly on vessel growth (e.g., CM-101, which is derived from exotoxin of Group A Streptococcus antigen and binds to new blood vessels inducing an intense host inflammatory response; and Thalidomide), a synthetic progesterone (e.g., medroxyprogesterone acetate (MPA), Oikawa (1988) Cancer Lett. 43: 85), a pro-drug of 5FU (e.g., 5′-deoxy-5-fluorouridine (5′DFUR), Haraguchi (1993) Cancer Res. 53: 5680-5682; Yayoi (1994) Int J Oncol. 5: 27-32; Yamamoto (1995) Oncol Reports 2:793-796), and polysaccharides capable of interfering with the function of heparin-binding growth factors that promote angiogenesis (e.g., pentosan polysulfate).

The “chemotherapeutic agent” includes chemical reagents that inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and Teicher, B. A. Cancer Therapeutics: Experimental and Clinical Agents (1996) Humana Press, Totowa, N.J. Other similar examples of chemotherapeutic agents include: bleomycin, docetaxel (Taxotere), doxorubicin, edatrexate, erlotinib (Tarceva), etoposide, finasteride (Proscar), flutamide (Eulexin), gemcitabine (Gemzar), genitinib (Irresa), goserelin acetate (Zoladex), granisetron (Kytril), imatinib (Gleevec), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

### Pharmaceutical Compositions

In one aspect, methods of administration of the invention are based on the administration of anti-tumor therapy (for example, in the form of cytotoxic agents or radiation) and an agent or treatment (for example, radiation) that modulates nitric oxide production in a solid tumor. In another aspect, methods of administration of the invention are based on the administration of anti-tumor therapy and an agent or treatment that modulates cGMP dependent protein kinase activity or expression in a solid tumor.

Thus, according to one embodiment of the present invention, a pharmaceutical composition is provided comprising a pharmaceutically acceptable carrier and a cytotoxic agent and/or an agent that modulates nitric oxide production. And in another embodiment, a pharmaceutical composition is provided comprising a pharmaceutically acceptable carrier and a cytotoxic agent and/or an agent that modulates cGMP dependent protein kinase activity or expression.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil, olive oil, and the like. Saline is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the cytotoxic or anti-angiogenic agent, in purified form, together with a suitable amount of carrier so as to provide the form for proper administration to the patient. The formulation should suit the mode of administration.

In an additional embodiment of the invention, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a suspending agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The amount of the pharmaceutical composition of the invention which will be effective in the treatment or prevention of a solid tumor will depend on the nature of the tumor and can be determined by standard clinical techniques. In addition, in vitro assays may optionally be employed to help identify optimal dosage ranges. The precise dose to be employed in the formulation will also depend on the route of administration, and the seriousness of the tumor, and should be decided according to the judgment of the practitioner and each patient's circumstances. Effective doses may be extrapolated from dose-response curves derived from in vitro or animal model test systems.

### Delivery/Administration

Various delivery systems are known and can be used to administer an agent or pharmaceutical composition of the present invention. For example, a non-viral delivery vehicle can be employed. “Non-viral delivery vehicle” includes chemical formulations containing naked or condensed polynucleotides (e.g., a formulation of polynucleotides and cationic compounds (e.g., dextran sulfate)), and naked or condensed polynucleotides mixed with an adjuvant such as a viral particle (i.e., the polynucleotide of interest is not contained within the viral particle, but the transforming formulation is composed of both naked polynucleotides and viral particles (e.g., adenovirus particles) (see, e.g., Curiel, et al. Am. J. Respir. Cell Mol. Biol. (1992)). Thus “non-viral delivery vehicle” can include vectors composed of polynucleotides plus viral particles where the viral particles do not contain the polynucleotide of interest.

“Non-viral delivery vehicles” include bacterial plasmids, viral genomes or portions thereof, wherein the polynucleotide to be delivered is not encapsidated or contained within a viral particle, and constructs comprising portions of viral genomes and portions of bacterial plasmids and/or bacteriophages. The term also encompasses natural and synthetic polymers and co-polymers. The term further encompasses lipid-based vehicles. Lipid-based vehicles include cationic liposomes such as disclosed by Feigner, et al (U.S. Pat. Nos. 5,264,618 and 5,459,127; PNAS 84:7413-7417, (1987); Annals N.Y. Acad. Sci. (1995); they may also consist of neutral or negatively charged phospholipids or mixtures thereof including artificial viral envelopes as disclosed by Schreier, et al. (U.S. Pat. Nos. 5,252,348 and 5,766,625).

Non-viral delivery vehicles include polymer-based carriers. Polymer-based carriers may include natural and synthetic polymers and co-polymers. Preferably, the polymers are biodegradable, or can be readily eliminated from the subject. Naturally occurring polymers include polypeptides and polysaccharides. Synthetic polymers include, but are not limited to, polylysines, and polyethyleneimines (PEI; Boussif, et al., PNAS 92:7297-7301, (1995)), which molecules can also serve as condensing agents. These carriers may be dissolved, dispersed or suspended in a dispersion liquid such as water, ethanol, saline solutions and mixtures thereof. A wide variety of synthetic polymers are known in the art and can be used.

Small delivery particles with cationic charge, e.g., high cationic charge, and larger size have recently been found to selectively target tumor vasculature, as compared with normal vessels. In one embodiment, the outer surface of the delivery particle is cationic at physiological pH. Such a charged outer surface may, for example, comprise a material selected from the group consisting of polyethylene glycol (PEG) (derivitized, e.g., to comprise a trimethyl ammonium moiety, a carboxylic acid moiety, a sulfonic acid moiety, or a hydroxyl group), N-(2-hydroxypropyl) methacrylamide (HPMA), poly(vinyl-pyrrolidone) (PVP), poly(ethyleneimine) (PED, a polyamidoamine, divinyl ether and maleic anhydride (DIVEMA (DIVEMA), dextran (α-1,6 polyglucose, dextrin (α-1,4 polyglucose), hyaluronic acid, a chitosan, a polyamino acid, poly(lysine) or poly(glutamic acid), poly(malic acid), poly(sapartamides), poly co-polymers, or copaxone.

Such delivery particles may be about 100-400 nm in diameter. So-called “nanoparticles” are multi-layered compositions for the delivery of therapeutic or diagnostic agents to a solid tumor that are not larger than about 300-400 nm in diameter. Such nanoparticles may comprise an inner core comprised of, for example, a polymeric substance comprising a diagnostic or therapeutic agent, or, have an inner core surrounded by a charged outer surface (as described above). Their charge and size can be adapted to some degree to allow delivery to the endothelium without penetration to the tumor cells. See, for example, PCT/US2006/038680, the contents of which are incorporated herein by reference.

Nucleic acids encoding recombinant agents of the invention (e.g., recombinant nitric oxide synthase, such as endothelial nitric oxide synthase, interfering RNA molecules that interact with target nNOS and iNOS RNA molecules) are inserted into delivery vectors and expressed from transcription units within the vectors. The recombinant vectors can be DNA plasmids or viral vectors (such as, but not limited to, retroviral, lentiviral, adenoviral, adeno-associated viral, pox viral, alphaviral). Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)).

Various techniques may be employed for introducing nucleic acids into cells. Such techniques include transfection of nucleic acid-CaPO₄ precipitates, transfection of nucleic acids associated with DEAE, transfection with a retrovirus including the nucleic acid of interest, liposome mediated transfection, and the like. Polymeric delivery systems also have been used successfully to deliver nucleic acids into cells, as is known by those skilled in the art. Such systems even permit oral delivery of nucleic acids.

A specific method of introducing nucleic acids of the invention into cells is by transducing cells using replication-deficient retroviruses. Replication-deficient retroviruses are capable of directing synthesis of all virion proteins, but are incapable of making infectious particles. Accordingly, these genetically altered retroviral vectors have general utility for high-efficiency transduction of genes. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art. The major advantage of using retroviruses is that the viruses insert efficiently a single copy of the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types.

Yet another viral candidate useful as an expression vector for transformation of cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. However, certain adenoviral sequences can confer intrachromosomal integration specificity to carrier sequences, and thus result in a stable transduction of the exogenous genetic material.

A variety of suitable vectors are available for transferring exogenous genetic material into cells. The selection of an appropriate vector to deliver a nucleic acid of the invention and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works contiguous with the coding sequence (in cis) to change the basal transcription level dictated by the promoter. Preferably, the exogenous genetic material is introduced into the cell genome immediately downstream from the promoter so that the promoter and coding sequence are operatively linked so as to permit transcription of the coding sequence. A preferred retroviral expression vector includes an exogenous promoter element to control transcription of the inserted exogenous gene. Such exogenous promoters include both constitutive and inducible promoters, and include promoters having specificity for tumor vasculature (e.g., to express nitric oxide synthase) as well as promoters having specificity for non-vascular cells of the tumor (e.g., to express interfering RNA).

Thus, as will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation.

Agents of the invention may be introduced into a subject through standard routes including, but not limited to, intradermal, intramuscular, intraperitoneal, intravenous, subcutaneous, intrathecal, intranasal, epidural, and oral routes. Methods of introduction may also be intra-tumoral (e.g., by direct administration into the area of the tumor).

The compositions may be administered by any convenient route, for example, by infusion or bolus injection, by absorption through epithelial or mucocutaneous linings (e.g., oral mucosa, rectal and intestinal-mucosa, etc.) and may be administered together with other biologically active agents (Jain, R., et al. 2006 Nature Clinical Practice Oncology 3(1):24-40). Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, by injection, by means of a catheter, or by means of an implant, said implant being of a porous, non-porous, or gelatinous material, including membranes, such as silastic membranes, or fibers. In one embodiment, administration can be by direct injection at the site (or former site) of a malignant tumor or neoplastic or pre-neoplastic tissue.

The cytotoxic agent and/or agent that modulates nitric oxide production may also be delivered in a controlled release system. In one embodiment, a pump may be used (see Langer, supra; Sefton, CRC Crit. Ref. Biomed. Eng. 14:201 (1987); Buchwald et al., 1980, Surgery 88: 507; and Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem.: 23:61 (1983); see also Levy et al., 1985, Science 228:190; During et al., 1989, Ann. Neurol. 25:351; and Howard et al., 1989, J. Neurosurg. 71:105). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (1990, Science 249:1527-1533).

The present invention also provides methods for treating a solid tumor comprising administering to a subject in need thereof, anti-tumor therapy (for example, in the form of cytotoxic agents or radiation) and an agent or treatment (for example, radiation) that modulates nitric oxide production. Thus, according to one embodiment of the present invention, a pharmaceutical composition is provided comprising a pharmaceutically acceptable carrier and a cytotoxic agent and/or an agent that modulates nitric oxide production. The present invention may include the sequential or concomitant administration of the anti-tumor therapy (in one embodiment, in a pharmaceutical composition) and an agent (likewise in a pharmaceutical composition, in one embodiment) or treatment that modulates nitric oxide production. The invention, thus, encompasses combinations of cytotoxic agents and/or radiation therapy and/or other nitric oxide production-modulating agents that are additive or synergistic.

In one embodiment, a subject with a solid tumor cancer is administered a pharmaceutical composition of the invention and treated with radiation therapy (e.g., gamma radiation or x-ray radiation). In a specific embodiment, the invention may, thus, provide a method to treat or prevent cancer that has shown to be refractory to radiation therapy. The pharmaceutical composition may be administered concurrently with radiation therapy.

The radiation therapy administered prior to, concurrently with, or subsequent to (though certainly within the “normalization window” of the tumor) the administration of the pharmaceutical composition of the invention can be administered by any method known in the art. Any radiation therapy protocol can be used depending upon the type of cancer to be treated. For example, but not by way of limitation, x-ray radiation can be administered; in particular, high-energy megavoltage (radiation of greater that 1 MeV energy) can be used for deep tumors, and electron beam and orthovoltage x-ray radiation can be used for skin cancers. Gamma ray emitting radioisotopes, such as radioactive isotopes of radium, cobalt and other elements may also be administered to expose tissues to radiation.

Administration of a therapeutic agent or treatment to the tumor vasculature can be selective with respect to its effect. The result of such selective administration is the provision of an effect (e.g., an increase in NO production) on eNOS alone, even if trace amounts of the agent or therapy in question is provided to non-vascular cells.

Likewise contemplated herein is co-administration of a therapeutic agent or treatment that effects an increase in eNOS activity or expression, along with an inhibitor of iNOS and/or nNOS to the tumor vasculature.

### Kits

The invention also provides a pharmaceutical pack or kit comprising one or more containers filled with one or more of the ingredients of the pharmaceutical compositions of the invention. Optionally associated with such container(s) can be a notice in the form prescribed by governmental agency regulating the manufacture, use or sale of pharmaceuticals or biological products, which notice reflects approval by the agency of manufacture, use or sale for human administration.

The present invention is additionally described by way of the following illustrative, non-limiting Examples that provide a better understanding of the present invention and of its many advantages.

### EXAMPLES

### Materials and Methods

### Cells and Transfection

U87MG (American Type Culture Collection HTB-14) and GL261 (generous gift from Dr. G. Yancey Gillespie at University of Alabama) tumor cells were cultured in Dulbecco's Modified Eagle's Medium with 10% fetal bovine serum at 37° C. in a humidified 5% CO₂ incubator. The following short-hairpin RNAs (shRNAs) for nNOS were designed using a commercial service program for small interfering RNA (siRNA) target finder (http://www.ambion.com/techlib/misc/siRNA_finder.html): nNOS-shRNA58, CAAAGAGATCGACACCATC (sense), GATGGTGTCGATCTCTTTGTT (antisense); nNOSshRNA97, CACGCATGTCTGGAAAGGC (sense), GCCTTTCCAGACATGCGTGTT (antisense); nNOS-shRNA150, GGTCTATCCAATGTCCACA (sense), TGTGGACATTGGATAGACCTT (antisense). The DNA oligonucleotides consist of a 19-nucleotide sense siRNA sequence linked to its reverse complementary antisense siRNA sequence by a short spacer (TTCAAGAGA).

Each DNA oligonucleotide was prepared with nucleotide overhangs with BamHI and HindIII restriction sites added to the 5′ and 3′ end of the DNA oligonucleotides and subcloned into the pSilencer 3.1H-1-hygro (Ambion) that allows transcription of the shRNA. These expression vectors were stably transfected into U87MG cells using LipofectAMINE 2000 (Invitrogen) following the manufacturer's instruction.

The stably transfected cells were selected with 80 μg/ml of hygromycin B. The expression of nNOS protein in U87 tumor cells and tissues was determined by Western blot analysis. Cultured U87MG cells washed with PBS and U87MG tumors homogenized by a tissue grinder were solubilized in 40 mM Tris-HCl, pH 6.8, 2% SDS, 10% glycerol, 1% β-mercaptoethanol, 0.01% bromophenol blue with one Complete Mini Protease Inhibitor Cocktail tablet (Roche Diagnostics) per 50 ml buffer.

Equal amounts of 60 micrograms of protein per sample were separated on 7.5% SDS polyacrylamide gels, transferred onto polyvinylidene fluoride membrane (Millipore), incubated with primary antibodies followed by secondary antibodies (the same antibodies used in immunohistochemistry studies. See Immunohistochemistry methods below), and detected using enhanced chemiluminescence (GE Healthcare) by exposure on autoradiography films (Kodak) (Xu, L., et al. 2005 Cancer Res. 65, 5711-5719; Carmeliet, P., et al. 1998 Nature 394, 485-490; Xu, L., et al. 2002 Journal of Biological Chemistry 277, 11368-11374 (2002); and Xu, L., et al. 2004 Clinical Cancer Research 10, 701-707). Animals and Tumor Models.

Recombination activating gene 1 (Rag-1^{-/-}) mice backcrossed to C57BL/6 background or severe combined immunodeficiency (SCID) mice, bred and maintained in an gnotobiotic animal facility, were used. To obtain source tumor tissue, U87 tumor cells in culture (1×10⁶ cells) were injected subcutaneously into Rag-1^{-/-} or SCID mice matching the recipient mice. When the tumor reached about 8 mm in diameter, it was excised after euthanasia and a small piece (about 1 mm³) of viable tumor tissue was implanted into a cranial window or subcutaneously into the calf area of the right hindlegs of the mice as previously described (Kashiwagi, S., et al. 2005 J. Clin. Invest. 115, 1816-1827; Kozin, S. V., et al. 2001 Cancer Research 61, 39-44). All animal procedures were carried out following the Public Health Service Policy on Humane Care of Laboratory Animals and approved by the Institutional Animal Care and Use Committee of Massachusetts General Hospital.

### NOS Inhibitors

To lower NO production from all NOS isoforms in the gliomas, mice received a pan-NOS inhibitor NG-Monomethyl-L-arginine monoacetate (L-NMMA) (Alexis Corp.) or non-active control compound D-NMMA (Alexis Corp.) at the rate of 7 mg/day by a constant release micro-osmotic pump (Model 1002, Alzet Osmotic Pumps, Durect Corp.) (Kashiwagi, S., et al. 2005 J. Clin. Invest. 115, 1816-1827). The micro-osmotic pumps were implanted in the back of the animals one day before the implantation of tumors. To selectively block nNOS activity, mice received an nNOS selective inhibitor NG propyl-L-arginine (L-NPA, Cayman Chemical) or saline control by daily intraperitoneal injection (20 mg/kg/day) started after the tumor implantation (Klamer, D., et al., 2004 European Journal of Pharmacology 503, 103-107). For selective eNOS inhibition, mice received a daily intraperitoneal injection of Cavtratin, a cell-permeable peptide derived from caveolin-1, at 2.5 mg/kg or the control peptide AP at 1.2 mg/kg, started after the tumor implantation (Gratton, J. P., et al. 2003 Cancer Cell 4, 31-39).

### Intravital Microscopy

Angiogenesis and vessel morphology were determined in U87 and GL261 tumors grown in the cranial windows by intravital microscopy using multiphoton laser scanning microscopy (MPLSM) or single photon fluorescence microscopy (SPFM) when tumors reached about 7 mm². Microangiography was performed after i.v. injection of 0.1 ml 10 mg/ml FITC or rhodamine-Dextran (2,000 kDa) as described previously (Kashiwagi, S., et al. 2005 J. Clin. Invest. 115, 1816-1827). Using MPLSM, five locations with 200-μm thick imaging stack were recorded for each tumor.

A semi-automated 3-D analysis system for blood vessels was used (Tyrrell, J. A., et al. 2007 IEEE Transactions on Medical Imaging 26, 223-237). Briefly, a superellipsoid was fitted into and passed along each visualized vessel, and each vessel was divided into short segments, for which length, diameter, position, and orientation were stored. From this data set, characteristics of the vasculature were calculated such as the total vessel length in a given 3-D volume and mean vessel diameter (length weighted). In some cases, five randomly selected locations of the tumors were imaged by SPFM.

Vascular parameters such as functional vessel density (the total length of perfused microvessels per unit area) and vessel diameter were analyzed by tracing each vessel segment using NIH image 1.63 as described elsewhere (Kashiwagi, S., et al. 2005 J. Clin. Invest. 115, 1816-1827). Tissue distribution of NO was visualized using MPLSM and the NO-sensitive fluorescence probe 4,5-diaminofluorescein (DAF-2) (0.5 mg i.v. Daiichi Pure Chemicals Co. Ltd) as described previously (Kashiwagi, S., et al. 2005 J. Clin. Invest. 115, 1816-1827). NO converts DAF-2 to 4,5-diaminofluorescein triazolium (DAF-2T) increasing fluorescence by a factor of 200. The DAF-2 associated fluorescence images were captured 60 min after i.v. injection. Known concentrations of DAF-2T were used for data calibration.

The effective vascular permeability (P) was determined by SPFM as described previously (Fukumura, D., et al. 2001 Proc Natl Acad Sci USA 98, 2604-2609). In brief, the fluorescence intensity of the tumor tissue was intermittently measured for 20 min after the injection of tetramethylrhodamine-labeled bovine serum albumin (10 mg/ml, 0.1 ml per 25 g body weight). Permeability was calculated as P=(1−HT) V/S [1/(I0−Ib)*dI/dt+1/K], where I is the average fluorescence intensity of the whole image, I0 is the initial fluorescence intensity, and Ib is the background fluorescence intensity, HT is hematocrit, V and S are the total volume and surface area of vessels within the tissue volume covered by the surface image, respectively, and K is the time constant of BSA plasma clearance (Fukumura, D., et al. 2001 Proc Natl Acad Sci USA 98, 2604-2609).

### Immunohistochemistry

To determine NOS expression, tumors were excised, fixed in 4% paraformaldehyde and embedded in paraffin. Sections (5 μm thick) were immunostained with antibodies to (1:1000), to nNOS (1:1000) or to iNOS (1:200) (all from BD Transduction Laboratory) and avidin-biotin complex/diaminobenzidine histochemistry as described (Kashiwagi, S., et al. 2005 J. Clin. Invest. 115, 1816-1827). Slides were analyzed using BX40 upright microscope (Olympus America Inc.).

To determine the extent of blood vessel coverage by perivascular cells, the tumor bearing mice were perfusion fixed with 4% paraformaldehyde following biotinylated lectin (Vector Laboratories) i.v. injection. Perfused blood vessels were stained with peroxidase-conjugated streptavidin (KPL) and visualized with true blue chromogen (KPL). Perivascular cells were identified using antibody to alpha smooth muscle actin (1:200, clone 1A4, Sigma) and alkaline phosphatase-conjugated secondary antibodies (DAKO). Fast Red (DAKO) served as substrate for alkaline phosphatase to visualize pericytes. Digital images of the immunohistochemistry slides were taken and analyzed as described (Kashiwagi, S., et al. 2005 J. Clin. Invest. 115, 1816-1827).

The percent of perivascular cell positive segments was determined in each vessel perimeter using NIH image 1.63 as described elsewhere (Kashiwagi, S., et al. 2005 J. Clin. Invest. 115, 1816-1827). Five locations from each tumor were randomly sampled and analyzed three to four tumors per group.

To evaluate tumor hypoxia, a hypoxia marker pimonidazole was used (Winkler, F., et al. 2004 Cancer Cell 6, 553-563). Briefly, when tumors grown in the hindleg reached about 100 mm³, 60 mg/kg pimonidazole was injected i.v. into the mice bearing subcutaneous tumors (100 mm³) 1 hr before perfusion fixation following biotinylated lectin injection. Blood vessels were stained with a Alexa 488-conjugated streptavidin (Vector Laboratory). Pimonidazole-adducts in hypoxic cells were detected using Hypoxyprobe-1 kit (Millipore) stained with tetramethylrhodamine isothiocyanate (TRITC)-conjugated goat antibody to mouse IgG. Cell nuclei were counter-stained by 4,6-diamidino-2-phenylindole (DAPI). Fluorescent images were taken using confocal laser-scanning microscopy, and vessel density and the pimonidazole-positive hypoxic area were determined using NIH image 1.63 macro (Winkler, F., et al. 2004 Cancer Cell 6, 553-563).

The macro identified TRITC-pimonidazole positive area and Alexa 488-positive perfused vessel area and binarized them at the same threshold. The percent of pimonidazole positive area to the total area was determined, and then the number and the perimeters of the binarized vascular areas were quantified on image J software. These parameters were determined in three-five photographic areas from each tumor (630×630 μm² each).

### Radiotherapy

When tumors grown in the hindleg reached about 100 mm³, animals were randomly assigned to radiation or control group (day 0). In the radiation groups, tumors were locally irradiated with three daily fractions, 8 Gy each, using a ¹³⁷Cs gamma irradiator (Hranitzky, E. B., et al. 1973 Radiology 107, 641-644) at a dose rate of approximately 5 Gy/min. The details of irradiation are described elsewhere (Kozin, S. V., et al. 2001 Cancer Research 61, 39-44). Tumor size was measured with a caliper at least every other day. The time taken for tumor to increase in volume 2×, 3×, 4×, 5×, 6×, and 7× of the initial volume (V₀) was determined. Tumor growth delay was calculated as the difference of this parameter between irradiated tumors and nonradiated control tumors of the same genotype. Mice were euthanized when the tumors reached 12 mm in diameter. Survival time of individual animals (spontaneous death or by euthanasia at maximum tumor size) was determined after the start of radiation/control treatment.

### Tumor Cell Radiosensitivity

The intrinsic radiosensitivity of wild-type and nNOS-shRNA transfected U87 cells was evaluated by performing survival curve assays using colony formation as an end point as previously described (Gerweck, L. E, et al. 1994 International Journal of Radiation Oncology, Biology, Physics 29, 57-66). Suspensions of single cells were prepared, counted, plated and irradiated in 25-cm² tissue culture flasks. The number of plated cells was adjusted to yield approximately 20-200 colonies per flask. Four-five flasks per dose were prepared. Lethally irradiated feeder cells (20 Gy) of the same genotype were added to yield a constant number of total cells per flask. The cells were irradiated 18-20 h following plating with 0-10 Gy in 2 Gy increments (Gamma-cell-40 137Cs Unit; Atomic Energy of Canada, Ltd.). The cells were then cultured for 9-13 d depending upon the dose administered; fixed with methanol and stained with crystal violet. The multiplicity corrected surviving fractions were calculated as the ratio of colonies (>50 cells) produced to the number of cells plated in irradiated vs. control flasks.

### Statistics

Unless otherwise specified the data were analyzed by unpaired Student's t-test using JMP™ (SAS Institute Inc.) when F test showed equality of variances. Values are expressed as mean±s.e.m. unless otherwise specified. Statistical significance was set at P<0.05.

### Example 1

### Re-Establishment of NO Gradient Normalizes Tumor Vasculature

It has previously been shown that endothelial nitric oxide synthase (eNOS) in vascular endothelial cells mediates recruitment of perivascular cells and maturation of blood vessels in both murine melanomas and tissue engineered blood vessels (Kashiwagi et al., JCI 2005). Human gliomas frequently express neuronal isoform of NOS (nNOS). NO production from glioma cells via nNOS would disrupt tissue gradient of NO from vascular endothelial cells and, thus, adversely affect perivascular cell recruitment and vessel maturation. Inhibition of nNOS in glioma cells may restore tissue gradient of NO from vascular endothelial cells and normalize tumor vasculature.

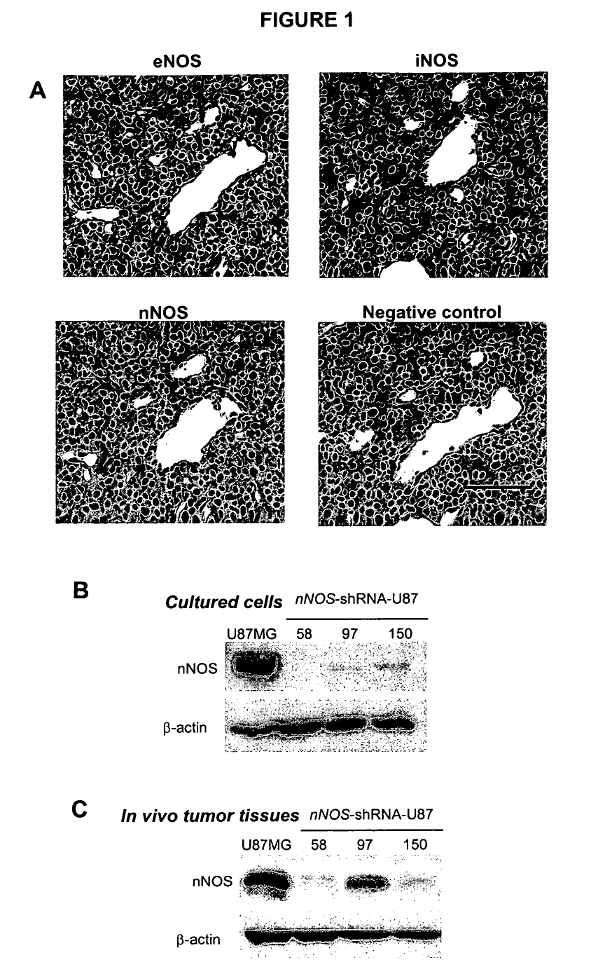

To test the NO-gradient hypothesis in vivo, the U87MG glioma model in which NO is produced by nNOS in tumor cells and eNOS in vascular endothelial cells was used (FIG. 1A). U87 human glioma cells, which express nNOS constitutively, were transfected with nNOS shRNA. To eliminate non-vascular NO production, three distinct populations of stably transfected U87 cells were established, each with a different design of nNOS shRNA vectors (pSilencer 3.1H-1 hygro (Ambion)). All of the transfected cells showed almost the same growth rate as U87 parental cells in vitro (data not shown). Western blot analysis showed that all three U87 nNOS-shRNA cells showed more than 98% knockdown efficiency in vitro (FIG. 1B). Knockdown efficiency was 99% for shRNA58, 98% for shRNA97 and 99% for shRNA150.

Knockdown efficiency was then examined in tumors in vivo. U87 parental cells and U87 with nNOS shRNA (shRNA58, shRNA97, and shRNA150) were grown in cranial windows in Rag-1^{-/-} mice. Expression of nNOS in these tumors was determined by Western blot analysis. Knockdown efficiency of nNOS in vivo was 98% for shRNA58, 67% for shRNA97, and 96% for shRNA150. In subsequent studies U87-shRNA58 and U87-shRNA150 were used, because they maintained high knockdown efficiency in vivo (FIG. 1C). Cranial windows were implanted in immunodeficient mice using the procedures described previously (Yuan, F., et al. 1994 Cancer Res 54:4564-4568). Briefly, circular areas of skin, bone, and dura matter were removed, a small piece of source tumor tissue was implanted, and the window was sealed via circular glass cover slip. The cranial window provides an orthotopic environment for gliomas and allows longitudinal intravital observations without further surgical manipulation.

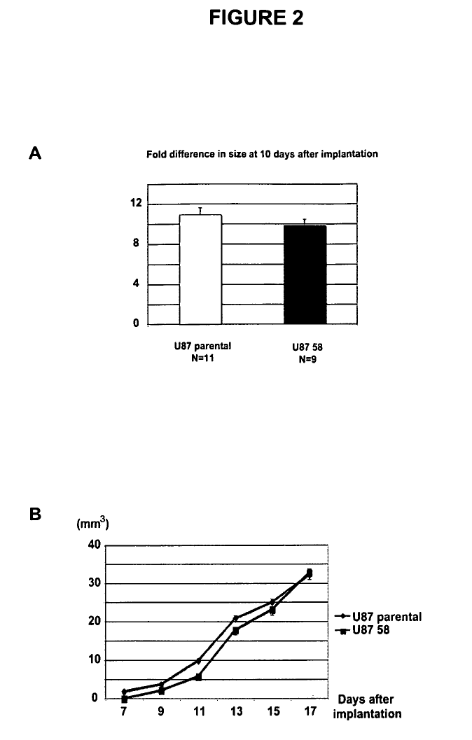

Subcutaneous tumors constitute a commonly used in vivo tumor model and were prepared by injection of tumor cells or transplantation of small tumor tissues into subcutaneous space in the hind leg of immunodeficient mice. The knockdown efficiency was observed to persist at least up to 14 days (in a cranial window model) or 21 days (in a subcutaneous model). Tumor growth rates were not different between parental and nNOS silencing tumors (FIG. 2).

### Example 2

### nNOS Silencing in Gliomas Reestablishes Tissue NO Gradient From Blood Vessels

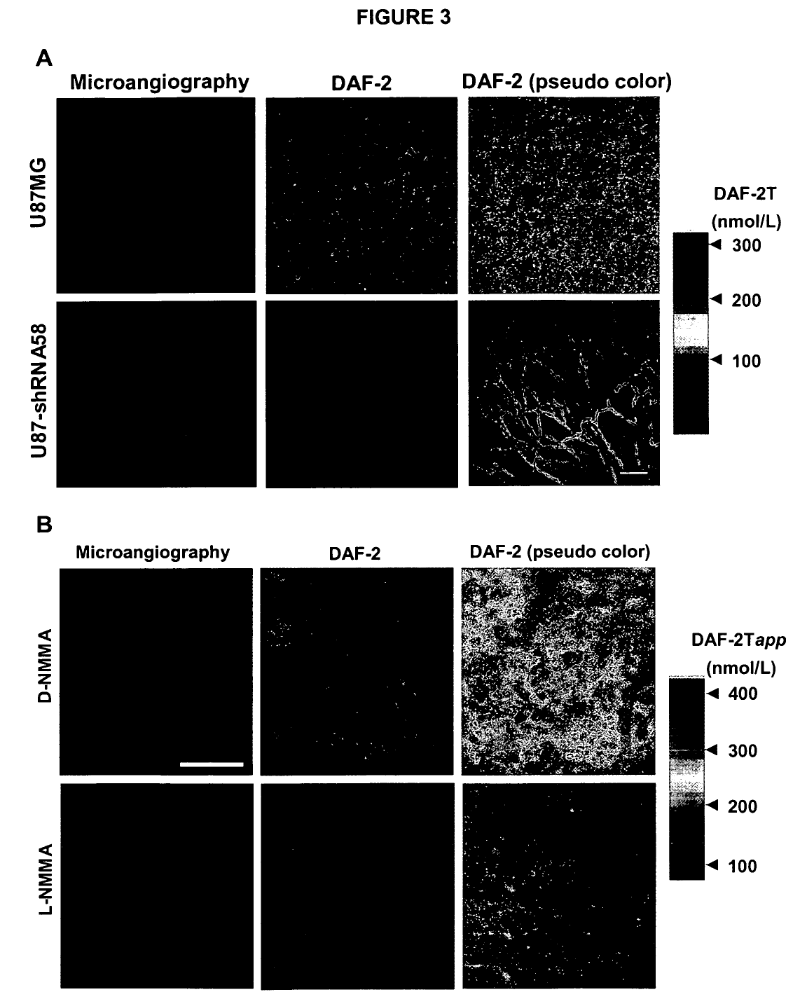

The distribution of NO in U87 tumors (parental and with shRNA58) was determined using DAF-2, an NO sensitive fluorescence probe (FIG. 3A). NO production was visualized by means of DAF-2T fluorescence using multi-photon laser-scanning microscopy (MPLSM) at 0, 20, 40 and 60 min after DAF-2 (0.5 mg/body) injection. DAF-associated fluorescence increased in a time-dependent manner both in vascular region and parenchyma in parental U87, as expected from the result of immunohistochemistry of NOSs (eNOS in blood vessels and nNOS in tumor cells). On the other hand, DAF-associated fluorescence predominantly localized in vascular region in U87 tumor with shRNA58, suggesting re-establishment of tissue NO gradient from blood vessels (FIG. 3A). DAF-associated fluorescence was abolished in animals treated with L-NMMA, an inhibitor of all NOS isoforms, compared to those treated with D-NMMA, a control compound (FIG. 3B).

### Example 3

### nNOS Silencing in Gliomas Facilitates Vessel Maturation

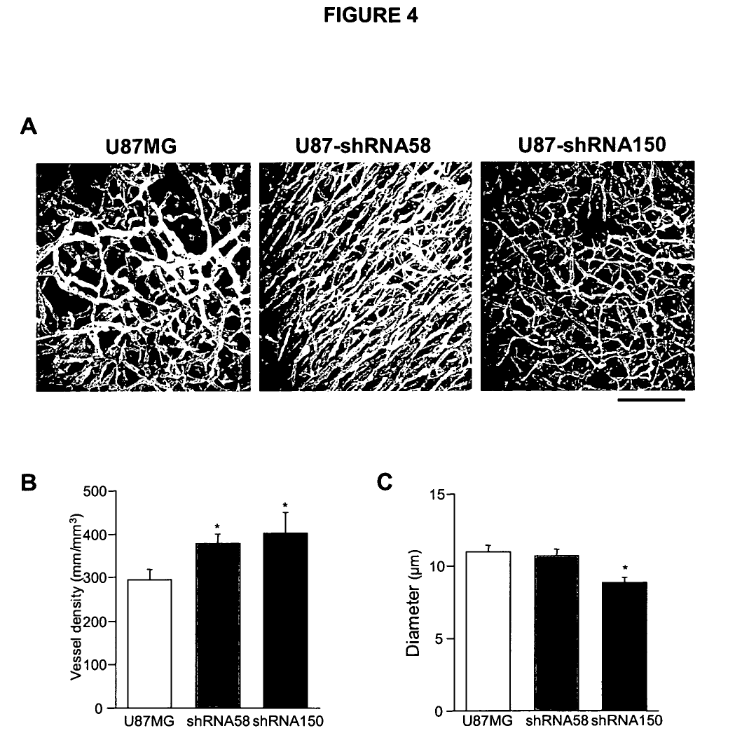

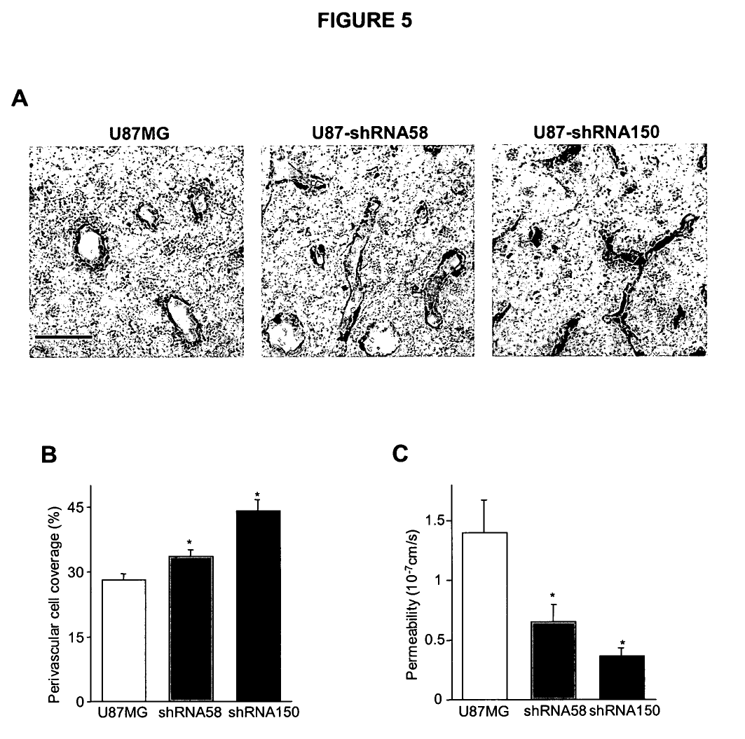

Microvascular parameters were determined by intravital microscopy in U87MG, U87-shRNA58, and U87-shRNA150 tumors grown in cranial window in Rag-1^{-/-} mice. U87 glioma in which nNOS is silenced had significantly higher vascular density compared to parental U87 tumors (FIG. 4B). Blood vessels were more evenly distributed and less tortuous in nNOS-silenced tumors as determined by intravital MPLSM (FIG. 4A). Average vessel diameter was also somewhat decreased in nNOS-silenced tumors (FIG. 4C). The association of perivascular cells with tumor blood vessels was subsequently determined by immunohistochemistry (FIG. 5A). On histological specimens of parental and nNOS silencing U87 gliomas, perivascular cells positive for the pericyte marker of a smooth muscle actin (αSMA) were identified. In the same section, perfused vascular endothelial cells were identified by injection of biotinylated lectin. The extent of pericyte coverage per vessel, as well as overall recruitment of perivascular cells was increased in nNOS silencing U87 tumors (FIG. 5B). Furthermore, nNOS silencing tumor had significantly smaller microvascular permeability (FIG. 5C), indicating a more mature phenotype of blood vessels.

Transfection of nNOS shRNA was, thus, shown to effectively and stably knock down nNOS expression in U87 glioma cells in vivo, re-establish tissue gradient of NO from vascular endothelial cells, and normalize tumor vasculature.

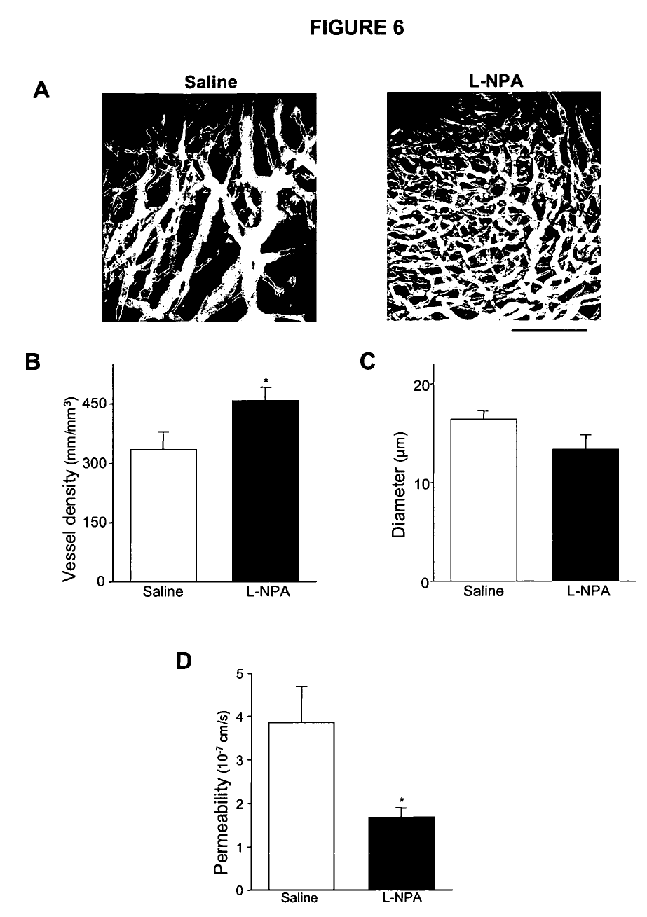

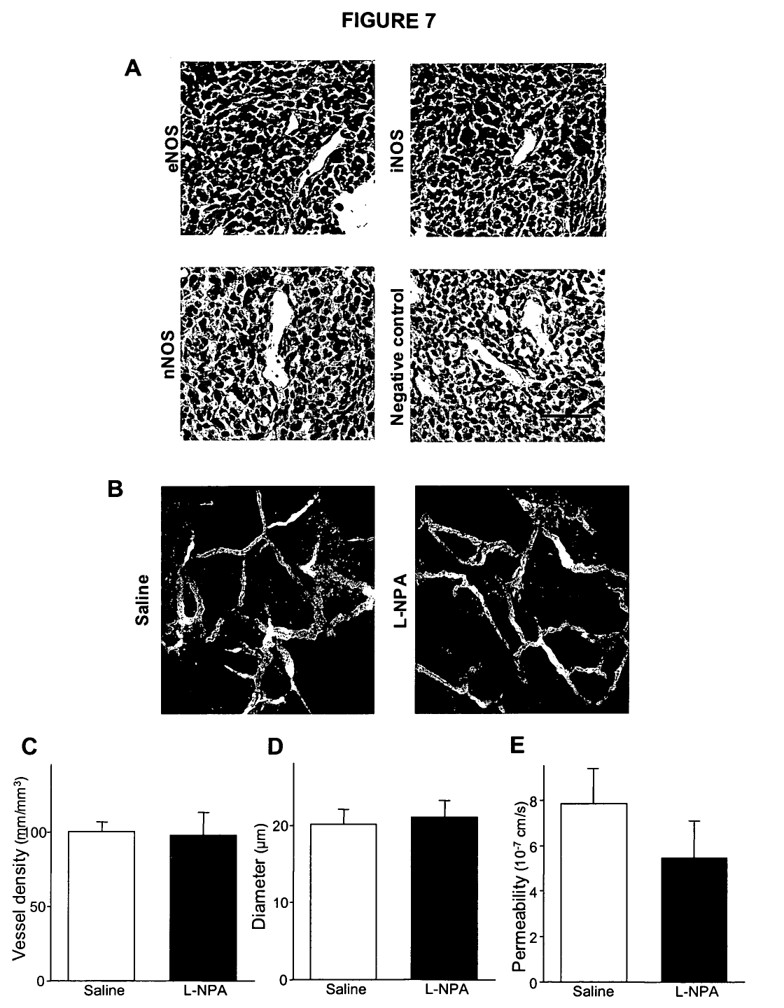

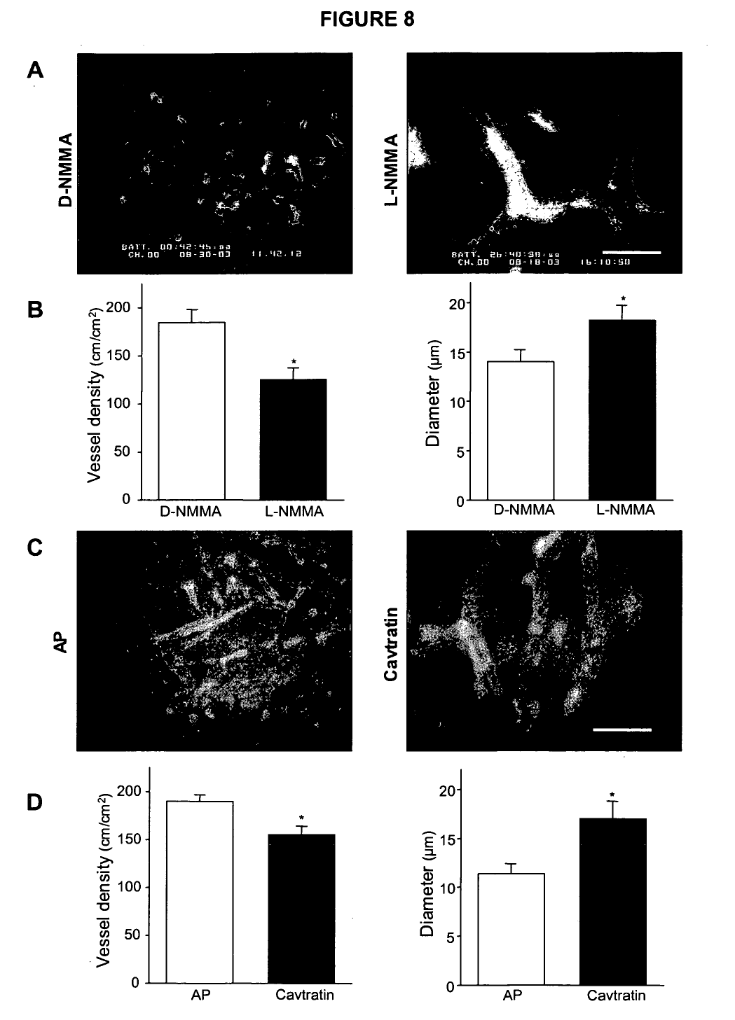

Treatment with an nNOS selective inhibitor, L-NPA, also increased vessel density and decreased vascular permeability in U87MG tumors grown in the cranial window (FIG. 6A-D). These results are in agreement with the vascular effects of nNOS silencing in tumor cells. However, this result was only specific for the nNOS selective inhibitor L-NPA in nNOS expressing tumors such as U87MG tumors. L-NPA did not improve tumor vasculature in tumor cells expressing other NOS isoforms such as eNOS and iNOS in GL261 tumors (FIG. 7A-E). Furthermore, in contrast to the elimination of non-vascular NO in U87MG tumors, decreased vessel density and increased vessel diameter in U87MG tumors were observed when vascular NO production was blocked by either cavtratin, a selective eNOS inhibitor, or L-NMMA a non-selective inhibitor of all NOS isoforms (FIG. 8A-D). These data indicate that the spatial distribution of NO, more specifically perivascular NO gradients, play a role in NO-mediated angiogenesis and stabilization of blood vessels in these tumors.

### Example 4

### nNOS Silencing in Gliomas Improves Tumor Oxygenation

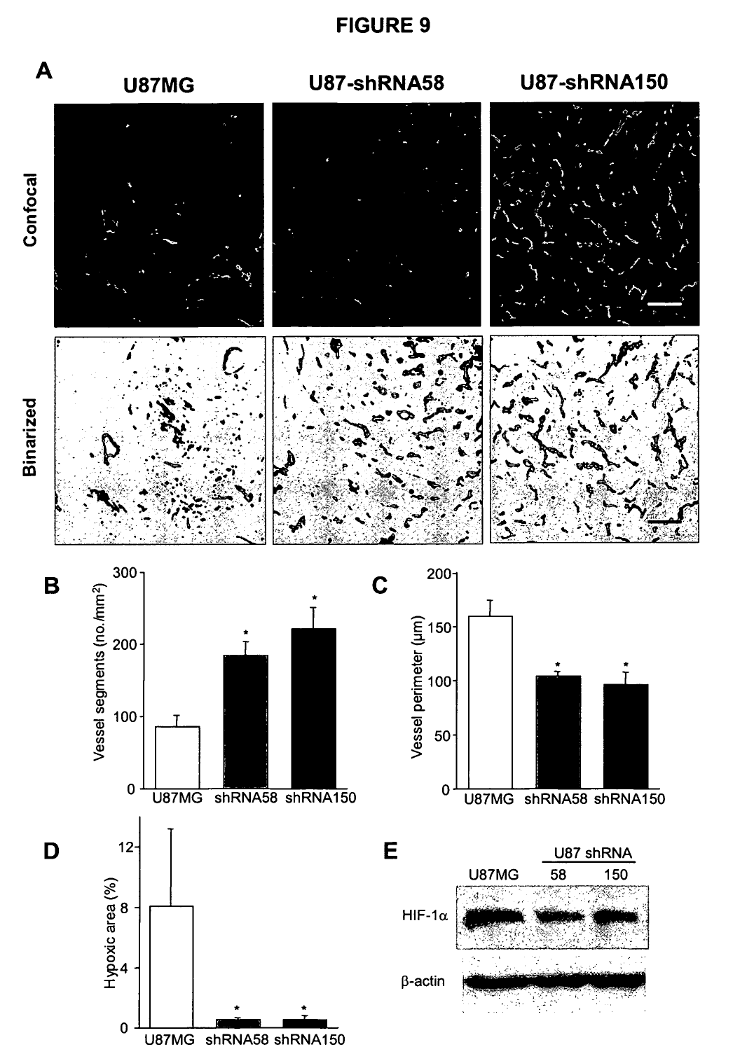

The extent of hypoxia was determined in U87MG, U87-shRNA58, and U87-shRNA150 tumors utilizing a redox marker pimonidazole (Hypoxyprobe™-1 Kit, Millipore). To determine the level of hypoxia during a commonly used experimental radiation treatment, tumors were grown to ˜100 mm³ subcutaneously in the hind leg of Rag-1^{-/-} mice. Immunofluorescence staining for hypoxia (Hypoxyprobe™-1 adducts) was observed in U87MG tumors, typically in the area distant from blood vessels (FIG. 9A). In nNOS silenced U87-shRNA58 tumors (U87-shRNA58 and U87-shRNA150), most of tumor cells have blood vessels in close proximity, and positive staining for hypoxia was hardly seen (FIG. 9A). In agreement with previous intravital observations in the cranium, increased vessel density was found in U87-shRNA58 and U87-shRNA150 tumors, as compared to U87MG tumors (FIG. 9B-C). Improved vascular morphology and function induced by the selective vascular localization of NO alleviate tumor tissue hypoxia (FIG. 9D). A reduction in hypoxia inducible factor-1α (HIF-1α) protein levels was also observed in the U87-shRNA58 and U87-shRNA150 tumors, as compared to U87MG tumors (FIG. 9E). These results indicated improved tissue oxygenation in nNOS-silenced tumors.

### Example 5

### nNOS Silencing in Gliomas Improves Response to Radiation Treatment

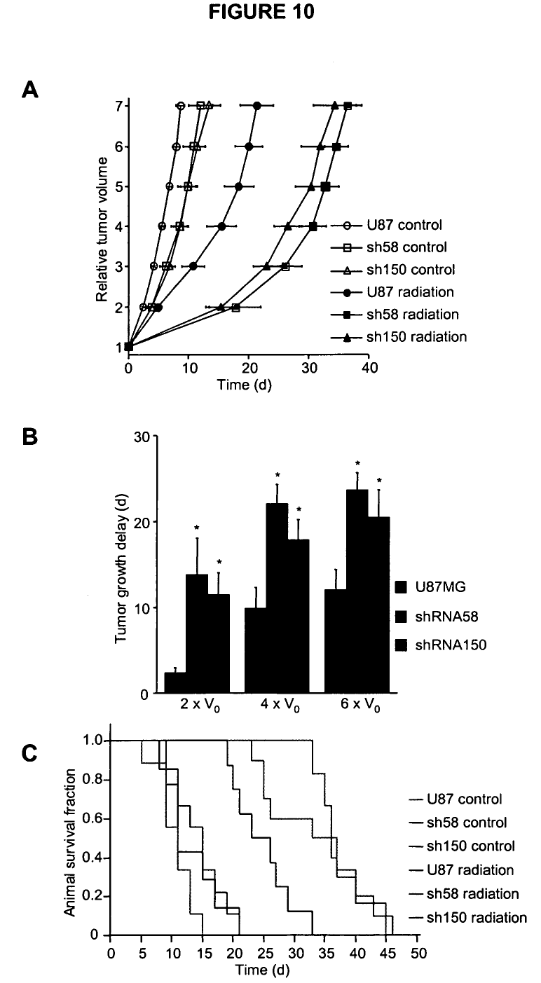

The effect of radiation treatment on U87MG, U87-shRNA58 and U87-shRNA150 tumors was determined. Tumors were grown subcutaneously in the hind leg of Rag-1^{-/-} mice. When tumors reached ˜100 mm³, they were randomly assigned to control and radiation treatment groups. Tumors were irradiated with daily fractions (8 Gy per fraction) on 3 consecutive days. Tumor growth and overall animal survival were monitored. Three daily fractions of 8 Gy-irradiation strongly suppressed tumor growth in nNOS-silenced U87 tumors while the effect on control U87MG tumor was modest (FIG. 10A). As it appears in FIG. 10A, tumor growth delay (difference from the corresponding untreated control tumors) by the fractionated radiation treatment was significantly longer in nNOS silencing tumors, as compared to parental U87MG tumors. Tumor growth delay by fractionated radiation treatment was significantly longer in nNOS-silenced tumors, compared to wild-type tumors (FIG. 10B).

Animals were euthanized when the tumors reached 12 mm in diameter. Significant extension of animal survival was observed in radiation treated U87-shRNA58 and U87-shRNA150 tumor bearing animals, as compared to the U87MG tumor bearing animals with radiation treatment (FIG. 10C). On the other hand, there was no significant difference in tumor growth and survival between non-irradiated nNOS-silenced and control U87MG tumors.

Thus, it is shown that transfection of nNOS shRNA effectively and stably knocks down nNOS expression in U87 glioma cells in vivo, re-establishes tissue gradient of NO from vascular endothelial cells, normalizes tumor vascular structure and function, and improves tissue oxygenation. Furthermore, response to radiation therapy is significantly improved in nNOS silencing tumors. These data indicate that modulation of NO signaling constitutes a viable strategy to normalize tumor vasculature and improve tumor treatments.

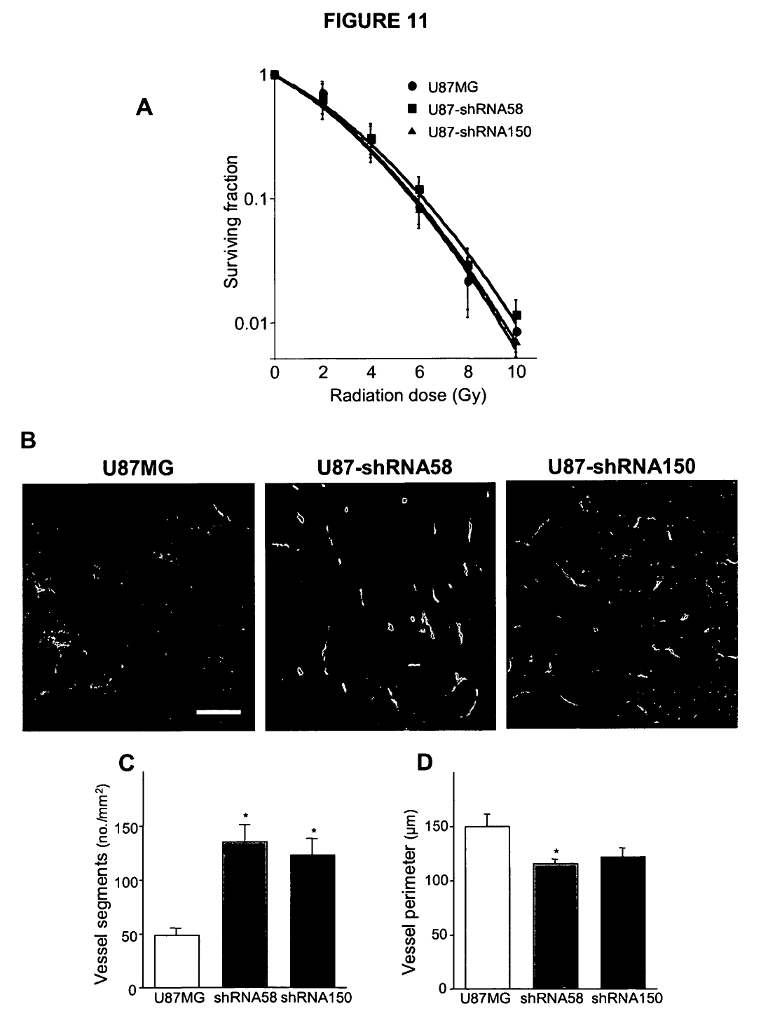

Oxygen significantly increases the radiation sensitivity of cells and tissues (Gerweck et al., 1994 International Journal of Radiation Oncology, Biology, Physics 29:57-66). In view of the oxygenation data in Example 4, several potential mechanisms that might contribute to the enhanced radiation response of nNOS silenced tumors were tested. Neuronal NOS silencing in itself did not alter the tumor cells' radiosensitivity (FIG. 11A). Nor could a report indicating that NO exposure sensitizes hypoxic cells to radiation explain the increased tumor response in NO silenced tumors (Mitchel, J. B., 1993 Cancer Res. 53, 5845-5848). Diffuse detection of NO-sensitive fluorescence signal in wild-type U87MG tumors (FIG. 3A) suggests that the uncoupled production of reactive oxygen radical species by nNOS may not be substantial (Pou S., et al. 1992 J. Biol. Chem. 267, 24173-24176). Additionally, there was no preferential vessel damage by radiation in nNOS silenced tumors (FIG. 11B-D). Thus, the increased radiation-induced tumor growth delay in nNOS-silenced tumors is most likely due to reduced tumor cell hypoxia. Collectively, these data indicate that creation of perivascular NO gradients is an effective strategy to improve tumor vasculature, drug delivery and oxygenation in tumors, and thus, response to various co-administered treatments.

### Example 6

### iNOS Inhibition Improves Tumor Vascular Function in Murine Breast Cancers

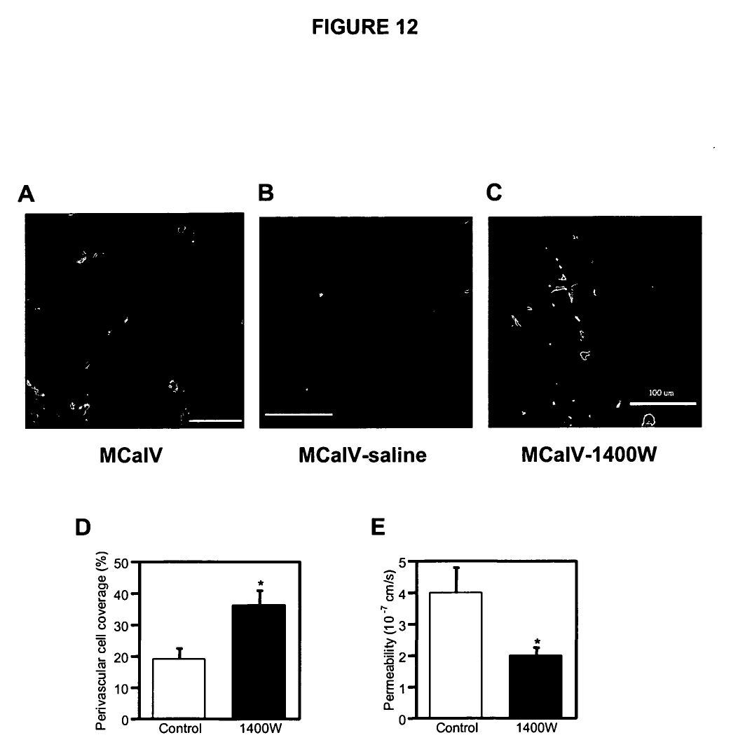

Breast cancer cells in MCaIV murine mammary tumor model express iNOS similar to many human tumors, in contrast to the U87MG glioma model, in which tumor cells predominantly express neuronal NOS (nNOS). MCaIV tumors grown orthotopically in the mammary fat pad express iNOS and to a much lesser extent eNOS as determined by real time RT-PCR. Immunohistochemistry revealed diffuse expression of iNOS in MCaIV tumor cells and in some macrophages (FIG. 12A). Inhibition of iNOS would eliminate the majority of non-vascular NO production. However, vascular NO production via eNOS would be preserved, and thus, establish selective vascular NO localization in MCaIV tumors. To block iNOS, MCaIV tumor bearing animals were treated with iNOS selective inhibitor 1400 W (10 mg/kg/day) using a constant release osmotic pump. To determine the effect of iNOS blockade on tumor vasculature, intravital microscopy was performed on MCaIV tumors grown in mouse dorsal skin chambers. αSMA^{P}-GFP mice (transgenic mice expressing green fluorescent protein under the control of a smooth muscle actin promoter) were used to assess perivascular cell coverage in real time using intravital microscopy. In the control tumors, tumor vessels were abnormally dilated, tortuous and leaky and exhibited sparse GFP-positive perivascular cells (FIG. 12B). Blockade of iNOS increased perivascular cell coverage (FIG. 12C). Quantification of the coverage of GFP positive perivascular cells over the functional vessel area revealed significantly increased perivascular cell coverage in MCaIV tumors treated with the iNOS inhibitor compared to saline treated control (FIG. 12D). The vascular permeability in control MCaIV tumors was high and increased with tumor growth. Administration of iNOS inhibitor prevented the increase in vascular permeability and that vascular permeability was significantly lower in iNOS inhibitor treated tumors as compared to the control tumors (FIG. 12E). These data indicate structural and functional improvement in MCaIV tumor vasculature by iNOS blockade.

### Example 7

### NO-sGC-cGMP Pathway Mediates Recruitment of Perivascular Cells to Vasculature

It has previously been shown that NO derived from vascular endothelial cells mediates recruitment of perivascular cells and maturation of blood vessels in both murine melanomas and tissue-engineered blood vessels (Kashiwagi et al., JCI 2005). NO activates soluble guanylyl cyclase (sGC), and sGC converts GTP to cGMP. cGMP regulates cell motility and contractility through various downstream signaling pathways such as PKG, MAPK and cGMP-gated cation channel. The sGC-cGMP pathway may mediate NO-induced perivascular cell recruitment and, thus, enhancing the sGC-cGMP pathway in perivascular cells may potentiate tumor vascular normalization.

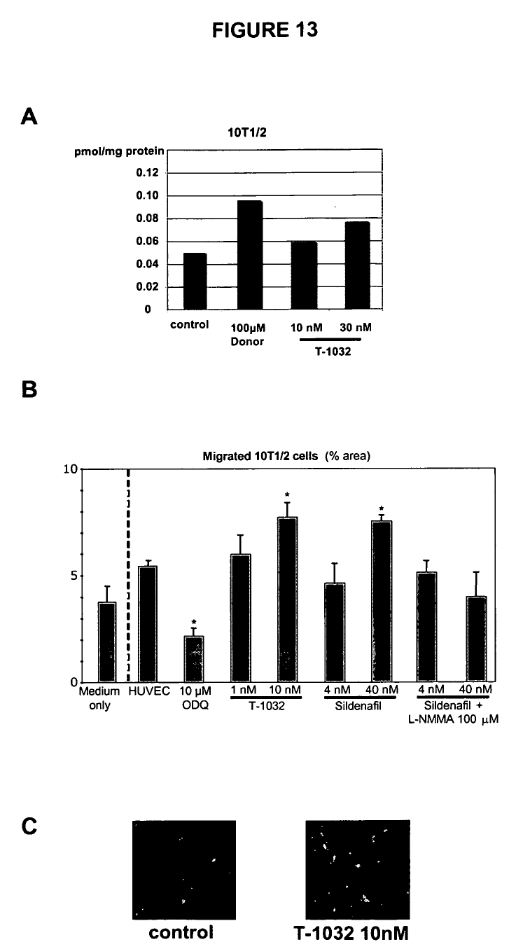

cGMP levels were examined in 10T1/2 cells following treatments of an NO donor DETANONOate or an inhibitor for phosphodiesterase 5 (PDE5), an enzyme which degrades cGMP. As shown in FIG. 13A, application of NO donor (100 μM DETANONOate) or a PDE5 inhibitor T-1032 (30 nM) increased cGMP level in 10T1/2 cells.

The effect of sGC inhibitor was next examined on 10T1/2 cell migration. As shown in FIG. 13B, 10 μM ODQ (an inhibitor of sGC) significantly reduced 10T1/2 cell migration to HUVECs (Mann-Whitney U-test). Finally, the effect of PDE5 inhibitors on 10T1/2 cell migration was examined. Application of PDE5 inhibitors such as T-1032 (10 nM) or Sildenafil (40 nM) significantly enhanced 10T1/2 cell migration (FIG. 13B-C). L-NMMA abolished induction of 10T1/2 cell migration induced by Sildenafil. (Mann-Whitney U-test).

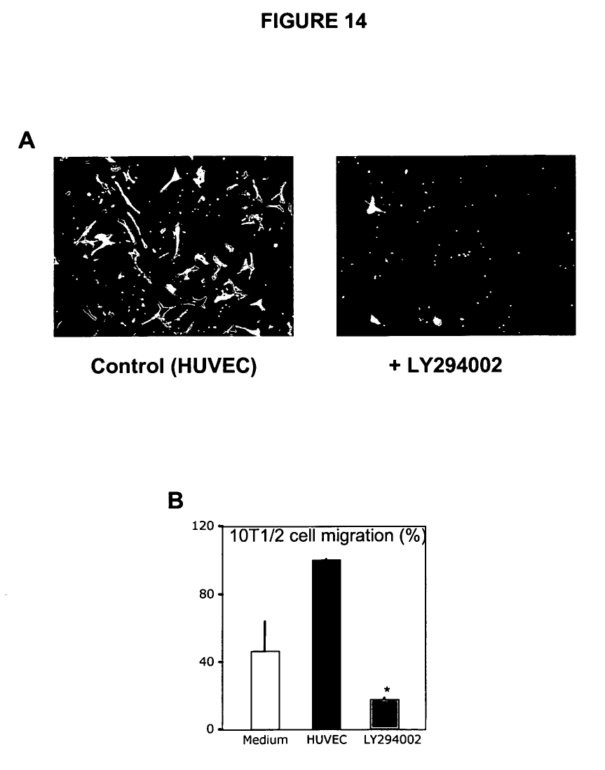

Because NO-mediated recruitment of perivascular cells was observed and it was found that sGC inhibitor can block migration of 10T1/2 cells (perivascular cell precursor) toward HUVECs, downstream signaling of the NO-sGC-cGMP-PKG pathway was further examined. To elucidate the downstream signaling pathway of NO-dependent perivascular cell recruitment, the effect of PI3K inhibitor LY291002 (10 μM) on the transwell 10T1/2 cell migration toward HUVECs was determined (FIG. 14A-B). LY291002 significantly inhibited migration of 10T1/2 cells indicating that PI3K mediates migration of perivascular cell precursors induced by NO-sGC-cGMP pathway.

### Example 8

### sGC is Highly Expressed in Perivascular Cells in Tumors

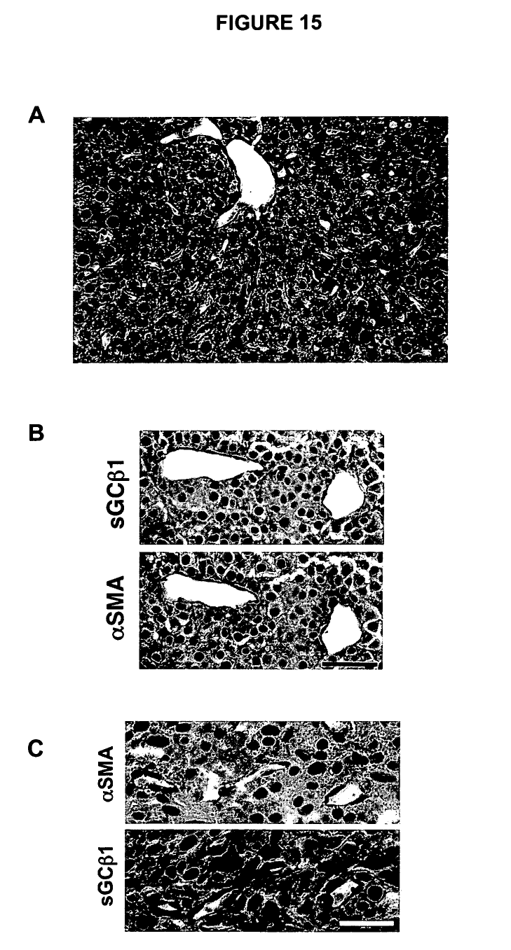

The expression of sGC was examined in solid tumors (B16F10 melanoma and U87 glioma). In normal tissue, sGC is expressed in local pericytes and considered to regulate pericyte contractility. Perfused sinusoids were stained by injection of biotinylated tomato-lectin and AP/Fast Red, and sGC was stained with anti-sGC antibody using the HRP-labeled polymer/DAB method. sGC expressing cells store fat droplets that are specific for hepatic stellate cells. As shown in FIG. 15A, hepatic stellate cells (liver specific pericytes) abundantly express sGC in situ.

Next, paraffin-embedded block sections were immunostained using anti-sGCβ1 (Cayman chemicals), αSMA antibody (Sigma) using the HRP-labeled polymer/DAB method. In B16F10 melanomas, sGC is selectively expressed in perivascular cells along tumor vessels (FIG. 15B).

In U87 gliomas, both tumor cells and perivascular cells express sGC. (FIG. 15C). These results indicate that perivascular cells in tumors can respond to NO, and that NO-induced morphogenesis of tumor vasculature is mediated by NO-sGC signaling pathway in perivascular cells.

Thus it has been shown herein that the sGC-cGMP pathway mediates NO-induced perivascular cell recruitment towards vascular endothelial cells in in vitro system, and that tumor perivascular cells express sGC.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method of treating a solid tumor in a subject, the method comprising the steps of:
modulating nitric oxide production in the tumor to normalize tumor vasculature;
and administering an anti-tumor therapy to the subject, thereby treating the solid tumor in the subject.

2. The method of claim 1, wherein the solid tumor is a glioblastoma.

3. The method of claim 1, wherein the nitric oxide production is selectively increased in the tumor vasculature.

4. The method of claim 3, wherein the nitric oxide production is selectively increased in the tumor vasculature by administering an agent that increases the expression of endothelial nitric oxide synthase to the tumor vasculature of the subject.

5. The method of claim 3, wherein the nitric oxide production is selectively increased in the tumor vasculature by administering an agent that increases the activity of endothelial nitric oxide synthase to the tumor vasculature of the subject.

6. The method of claim 5, wherein the agent that increases the activity of endothelial nitric oxide synthase is not expressed in the tumor, and wherein the agent is a peptide selected from the group consisting of a vascular endothelial growth factor, angiopoietin-1, platelet derived growth factor-beta, transforming growth factor-beta, estrogen, BH4: (6R)-5,6,7,8-tetrahydro-L-biopterin, RANKL, an inhibitor of caveolin-1 and bradykinin.

7. The method of claim 5, wherein the agent that increases the activity of endothelial nitric oxide synthase is selected from the group consisting of a statin, L-arginin, calcium ionophore, sphingosine-1-phosphate, nitrite and acethylcholine.

8. The method of claim 7, wherein the statin is selected from the group consisting of Atorvastatin, Cerivastatin, Fluvastatin, Lovastatin, Mevastatin, Pravastatin, Rosuvastatin and Simvastatin.

9. (canceled)

10. The method of claim 4, wherein the agent is administered in a cationic delivery vehicle that is greater than about 100 nm.

11. The method of claim 3, wherein the nitric oxide production is selectively increased in the tumor vasculature by providing low dose radiation in a range of between about 2 Gy to about 6 Gy to the subject.

12. The method of claim 3, wherein nitric oxide production is selectively increased in the tumor vasculature by administering endothelial nitric oxide synthase to the tumor vasculature of the subject.

13. The method of claim 12, wherein the endothelial nitric oxide synthase is administered in a cationic delivery vehicle that is greater than about 100 nm.

14. (canceled)

15. The method of claim 3, wherein nitric oxide production is selectively increased in the tumor vasculature by administering an expression vector comprising a nucleic acid sequence encoding an endothelial nitric oxide synthase to the tumor vasculature of the subject.

16. The method of claim 15, wherein the nucleic acid sequence encoding an endothelial nitric oxide synthase is expressed by an endothelial specific promoter.

17. (canceled)

18. The method of claim 3, wherein nitric oxide production is selectively increased in the tumor vasculature by administering a nitric oxide donor to the tumor vasculature of the subject.

19. The method of claim 18, wherein the nitric oxide donor is selected from the group consisting of a DETANONOate, GEA, SNAP, GSNO, ISDN, NOC, NOR, Spermine NONOate, NO-donating nonsteroidal anti-inflammatory drugs (NO-NSAIDs), nitrite and S-nitorosohemoblobin.

20. The method of claim 18, wherein the nitric oxide donor is administered by intravenous delivery.

21. The method of claim 18, wherein the nitric oxide donor is administered in a cationic delivery vehicle that is greater than about 100 nm.

22. The method of claim 1, wherein non-vascular cells of the tumor produce nitric oxide and said nitric oxide production is selectively decreased in said cells.

23-30. (canceled)

31. The method of claim 1, further comprising monitoring tumor vasculature to detect normalized tumor vasculature prior to administering the anti-tumor therapy to the subject.

32-66. (canceled)
